(19) United States
(12) Patent Application Publication
(10) Pub. No.: US 2021/0355492 A1
(43) Pub. Date: Nov. 18, 2021

(54) CARGO LOADED EXTRACELLULAR VESICLES

(71) Applicant: City University of Hong Kong, Kowloon (HK)

(72) Inventors: Jiahai Shi, Kowloon (HK); Thi Nguyet Minh Le, Kowloon (HK); Waqas Muhammad Usman, Kowloon (HK)

(73) Assignee: City University of Hong Kong, Kowloon (HK)

(21) Appl. No.: 17/278,275

(22) PCT Filed: Dec. 6, 2018
(86) PCT No.: PCT/SG2018/050596

§ 371 Date: Mar. 19, 2021

Related U.S. Application Data
(60) Provisional application No. 62/734,303, filed on Sep. 21, 2018.

(51) Int. Cl. C12N 15/113; A61K 35/18; C12N 15/11

(52) U.S. Cl. CPC C12N 15/113; A61K 35/18; C12N 15/111; C12N 2310/113; C12N 9/22; C12N 2310/321; C12N 2310/3231; C12N 2310/20; C12N 2310/315

(57) ABSTRACT

The application relates to extracellular vesicles derived from red blood cells and particularly, although not exclusively, extracellular vesicles derived from red blood cells containing a cargo. The cargo may comprise small molecules, proteins, nucleic acids or components of the CRISPR/Cas9 gene editing system. The extracellular vesicles derived from red blood cells may be used in the treatment of medical disorders such as a genetic disorder, inflammatory disease, cancer, autoimmune disorder, cardiovascular disease or a gastrointestinal disease. Also provided is a method for loading the cargo into the extracellular vesicles derived from red blood cells by electroporation.

This application claims priority from US 62/734303 filed 21 Sep. 2018 filed, the contents and elements of which are herein incorporated by reference for all purposes.

### FIELD OF THE INVENTION

The present invention relates to extracellular vesicles and particularly, although not exclusively, extracellular vesicles containing a cargo.

### BACKGROUND

RNA therapeutics including small-interfering RNAs (siRNAs), microRNAs (miRNAs), antisense oligonucleotides (ASOs), messenger RNAs (mRNAs), long non-coding RNAs and CRISPR-Cas9 genome editing guide RNAs (gRNAs) are emerging modalities for programmable therapies that target the diseased human genome with high specificity and flexibility. Common vehicles for RNA drug delivery, including viruses (e.g., adenoviruses, adeno-associated viruses, lentiviruses, retroviruses), lipid transfection reagents, and lipid nanoparticles, are usually immunogenic and/or cytotoxic. Thus a safe and effective strategy for the delivery of RNA drugs to most primary tissues and cancer cells, including leukemia cells and solid tumor cells, remains elusive.

Extracellular vesicles (EVs) have been applied to deliver RNA to patients. EVs are secreted by all types of cells in the body for intercellular communication. EVs comprised of exosomes which are small vesicles (10-100 nm) derived from the multivesicular bodies, microvesicles (100-1000 nm) derived from the plasma membrane of live cells and apoptotic bodies (500-5000 nm) derived from plasma membrane of apoptotic cells. EV-based drug delivery methods are desired but EV production has limitations. To produce highly pure and homogenous EVs, stringent purification methods such as sucrose density gradient ultracentrifugation or size exclusion chromatography are needed but they are time-consuming and not scalable. Moreover the yield is so low that billions of cells are needed to get sufficient EVs, and such numbers of primary cells are usually not available. Immortalization of primary cells would run the risk of transferring oncogenic DNA and retrotransposon elements along with the RNA drugs. In fact, all nucleated cells present some level of risk for horizontal gene transfer, because it is not predictable a priori which cells already harbor dangerous DNA, and which do not. Accordingly, there remains a need for effective approach for delivering nucleic acid material to patients with reduced side effects.

Further, to make EV-based therapy more specific, EVs may be engineered to have peptides or antibodies that bind specifically to certain target cell, by expressing peptides or antibodies in donor cells from plasmids that are transfected or transduced using retrovirus or lentivirus followed by an antibiotic based or fluorescence-based selection. These methods pose a high risk of horizontal gene transfer as the highly expressed plasmids are likely incorporated into EVs and eventually transferred to the target cells. Genetic elements in the plasmids may cause oncogenesis. If stable cell lines are made to produce EVs, abundant oncogenic factors including mutant DNAs, RNAs and proteins are packed in EVs and deliver to the target cells the risk of tumorigenesis. On the other hand, genetic engineering methods are not applicable to red blood cells as plasmids cannot be transcribed in red blood cells because of the lack of ribosomes. It is also not applicable to stem cells and primary cells that are hard to transfect or transduce.

Recently, there is a new method of coating EVs with antibodies fused to a C1C2 domain of lactadherin that bind to phosphatidylserine (PS) on the surface of EVs. This method allows conjugation of EVs with antibodies without any genetic modification. However, C1C2 is a hydrophobic protein and hence requires a tedious purification method in mammalian cells and storage in bovine serum albumin containing buffer. Moreover, the conjugation of EVs with C1C2-fusion antibodies is based on the affinity binding between C1C2 and PS that is transient.

Accordingly, there remains a strong need for a stable EV for therapeutic or diagnostic purpose. The present invention has been devised in light of the above considerations.

### SUMMARY OF THE INVENTION

Extracellular vesicles (EVs) are emerging drug delivery vehicles due to their natural biocompatibility, high delivery efficiency, low toxicity, and low immunogenic characteristics. EVs are usually engineered by genetic modifications of their donor cells however, genetic engineering methods are inefficient in primary cells and eventually post a risk of horizontal gene transfer which is unsafe for clinical applications. EV-mediated delivery of drugs including small molecules, proteins and nucleic acids is an attractive platform because of the natural biocompatibility of EVs that overcome most in vivo delivery hurdles. EVs are generally nontoxic and non-immunogenic. They are taken up readily by many cell types but they do possess some antiphagocytic markers such as CD47 that help them to evade the phagocytosis by macrophages and monocytes of the reticuloendothelial system. Moreover, EVs are able to extravasate well through the interendothelial junctions and even cross the blood-brain barrier hence, they are greatly versatile drug carriers.3 Of clinical value, delivery by EVs is not hampered by the multidrug resistance mechanism caused by overexpression of P-glycoproteins that tumor cells often use to eliminate many chemical compounds.

Accordingly, this disclosure relates to modified extracellular vesicles containing a cargo as well as methods for making and using such loaded extracellular vesicles.

At its most general, the disclosure provides an extracellular vesicle containing a cargo, referred to herein as a “loaded extracellular vesicle”. The cargo is preferably an exogenous material, meaning that it does not occur in the extracellular vesicles in nature, but has been introduced into the extracellular vesicles.

The disclosure relates particularly to extracellular vesicles derived from red blood cells. Such vesicles are distinct from extracellular vesicles derived from other cells, because they retain characteristics of red blood cells, such as pigmentation or presence of haemoglobin, or surface markers characteristic of a red blood cell such as CD235a. The membrane surrounding the vesicle may be characterised by the presence of one or more red blood cell surface markers, such as CD235a.

The cargo may be a nucleic acid, peptide, protein or small molecule. For example, the cargo may be a nucleic acid selected from the group consisting of an antisense oligonucleotide, a messenger RNA, a long RNA, a siRNA, a miRNA, a gRNA or a plasmid. In some cases, the nucleic acid comprises one or more modifications, non-naturally occurring elements or non-naturally occurring nucleic acids. The one or more modifications, non-naturally occurring elements or non-naturally occurring nucleic acids may be selected from a 2′-O-methyl analog, a 3′ phosphorothioate internucleotide linkage or other locked nucleic acid (LNA), an ARCA cap, a chemically modified nucleic acids or nucleotides or a 3′ or 5′ modification such as capping. The non-naturally occurring nucleotide may be selected from a 2′-position sugar modification, 2′-O-methylation, 2′-Fluoro modification, 2′NH2 modification, 5-position pyrimidine modification, 8-position purine modification, a modification at an exocyclic amine, substitution of 4-thiouridine, substitution of 5-bromo, or 5-iodo-uracil, or a backbone modification.

In particularly preferred aspects, the cargo comprises one or more components of a gene editing system, such as the CRISPR/Cas9 gene editing system. The cargo may be a nuclease, or an mRNA or plasmid encoding a nuclease. The cargo may comprise a gRNA.

In other preferred aspects, the cargo comprises an antisense nucleic acid or oligonucleotide. The cargo may be comprise a sequence complementary to a nucleic acid in a target cell or cell of interest. For example, an miRNA or an mRNA. In some embodiments, the nucleic acid is complementary to an oncogenic miRNA, such as miR125b.

Also disclosed herein are compositions comprising one or more extracellular vesicles loaded with a cargo. In some compositions the loaded extracellular vesicles are loaded with the same cargo. In other compositions, the loaded extracellular vesicles are loaded with two or more cargo molecules. Each extracellular vesicle may contain more than one different cargo. In some cases, a proportion of the extracellular vesicles in the composition contain one cargo, and another proportion of the extracellular vesicles contain a different cargo. In some cases, the proportions overlap, such that some extracellular vesicles in the composition are loaded with at least two different cargo molecules. Preferably, at least 75%, at least 80%, at least 85%, at least 90%, at least 95%, at least 97% or substantially all of the extracellular vesicles in the composition encapsulate a cargo.

The compositions may be useful in methods of treatment, in particular methods of medical treatment. The methods may involve administration of the composition to an individual in need of treatment.

Also described is the use of an extracellular vesicle or a composition comprising extracellular vesicles in the manufacture of a medicament of the treatment of a disease or disorder.

Extracellular vesicles described herein are useful for the treatment of a subject with a genetic disorder, inflammatory disease, cancer, autoimmune disorder, cardiovascular disease or a gastrointestinal disease. The cancer may be a leukemia, lymphoma, myeloma, breast cancer, lung cancer, liver cancer, colorectal cancer, nasopharyngeal cancer, kidney cancer or glioma.

Also described is a method comprising providing or obtaining a sample of red blood cells, wherein said sample is free from leukocytes; contacting the sample of red blood cells with a vesicle inducing agent; separating extracellular vesicles from red blood cells; and collecting the extracellular vesicles. The method may include a step of electroporating the extracellular vesicles in the presence of an exogenous cargo. Such a step may induce an extracellular vesicle to encapsulate or take up the cargo. The method may involve a step of treating a whole blood sample, or a sample containing red blood cells to remove non red blood cells, such as leukocytes or platelets from the sample. For example, by centrifugation and/or filtration.

Another method involves electroporating an extracellular vesicle derived from a red blood cell in the presence of an exogenous cargo. In this way an extracellular vesicle may be induced to encapsulate or take up the cargo.

The disclosure also contemplates extracellular vesicles and loaded extracellular vesicles obtained by the methods disclosed herein. Such extracellular vesicles are suitable for therapeutic use.

In preferred aspects, the cargo is loaded into the extracellular vesicle after the extracellular vesicle has formed. Preferably, the cargo is not loaded into to the extracellular vesicle during formation of that vesicle. The cargo may not be present in the cell from which the extracellular vesicle has been formed.

Also disclosed herein are extracellular vesicles and compositions containing extracellular vesicles used in medicine. Such compositions and extracellular vesicles may be administered in an effective amount to a subject in need of treatment. The subject may be in need of treatment for, or may have, a genetic disorder, inflammatory disease, cancer, autoimmune disorder, cardiovascular disease or a gastrointestinal disease. The cancer optionally selected from leukemia, lymphoma, myeloma, breast cancer, lung cancer, liver cancer, colorectal cancer, nasopharyngeal cancer, kidney cancer or glioma.

In a further aspect, there is provided a method of inhibiting the growth or proliferation of a cancer cell comprising contacting the cancer cell with an extracellular vesicle or composition according to the invention. Also disclosed herein are in vitro methods comprising contacting cell with an extracellular vesicle.

Methods of producing loaded extracellular vesicles are also disclosed herein, as well as extracellular vesicles obtained by such methods. At its most general, such methods involve contacting the extracellular vesicle with a cargo and electroporating to encapsulate the cargo with the extracellular vesicle. Methods of producing loaded extracellular vesicles may further include a step of purifying, isolating or washing the extracellular vesicle. Purifying, isolating or washing the extracellular vesicle may involve differential centrifugation of the extracellular vesicle. Differential centrifugation may involve centrifugation in a sucrose gradient, or a frozen sucrose cushion.

Methods disclosed herein include contacting a cell with a loaded RBC-EV wherein the method results in at least 60% of the contacted cells being transfected with the load.

In another aspect, disclosed herein is a method of preparing an extracellular vesicle suitable for therapeutic use, the method comprising isolating an extracellular vesicle from a red blood cell.

The invention includes the combination of the aspects and preferred features described except where such a combination is clearly impermissible or expressly avoided.

In one aspect of the invention, there is provided a method for RNA delivery to target cells comprising the steps of: a) purification of extracellular vesicles (EVs) from red blood cells (RBCs); b) electroporation of the EVs with RNAs to form RNA-loaded EVs; and c) applying the RNA-loaded EVs to the target cells.

The advantage of using EVs (including microvesicles and exosomes) from RBCs is that the RBCs are the most abundant blood cells hence a large amount of EVs can be obtained and purified from RBC units that are available at any blood bank. Preferably, the RBCs are derived from a human. They are also nontoxic, unlike synthetic transfection reagents. RBC EVs do not contain oncogenic DNA/RNA or growth factors that are usually abundant in EVs from cancer cells or stem cells, hence RBC EVs do not post any transformation risks to recipient cells.

In one embodiment, the RBCs are derived from a mammal preferably a human and treated with ionophore in particular calcium ionophore. The EVs are purified using ultracentrifugation with a sucrose cushion. The term “sucrose cushion” refers to a sucrose gradient which establishes itself during a centrifugation.

In an embodiment, the sucrose gradient is prepared by using a solution of about 40% to about 70%, about 50% to about 60%, or about 60% of sucrose. In another embodiment, the electroporated EVs comprises antisense oligonucleotides (ASO), mRNAs and plasmids. Preferably, the ASO comprises or consists of SEQ ID NO: 1.

In a further embodiment, the target cells comprise cancer cells, or are cancer cells. In another embodiment, the target cells comprise leukemia cells in particular acute myeloid leukemia (AML) cells, breast cancer cells, or a combination of AML cells and breast cancer cells.

In another embodiment, the EVs are electroporated with ASO antagonizing miR-125b for knockdown of miR-125b in target cells as described above. Preferably, the ASO antagonizing miR-125b comprises or consists of SEQ ID NO: 1.

In another embodiment, the growth of the target cells is suppressed.

In a further embodiment, the EVs are electroporated with a small chemical such as dextran. In another embodiment, the method comprises administering to the target cells the RNA loaded EVs which modulate an apoptosis-related gene expression, thereby inducing apoptosis in the target cells. In a second aspect of the invention, there is provided a method for delivery of an antisense oligonucleotide (ASO) to target cells to suppress gene expression, comprising the steps of: a) purification of extracellular vesicles (EVs) from red blood cells (RBCs); b) electroporation of the EVs with RNAs to form RNA-loaded EVs; and c) applying the RNA-loaded EVs to the target cells.

In an embodiment, as described above, the RBCs are derived from a mammal preferably a human, and treated with ionophore in particular calcium ionophore. In one embodiment, the RNA is an ASO antagonizing miR-125b to inhibit the oncogenic miR-125b in the target cells. Preferably, the ASO antagonizing miR-125b comprises or consists of SEQ ID NO: 1.

In another embodiment, the target cells comprise cancer cells or are cancer cells. In another embodiment, the target cells comprise leukemia cells in particular AML cells, breast cancer cells, or a combination of AML cells and breast cancer cells.

In a third aspect of the invention, there is provided a method of RNA delivery to target cells for a CRISPR genome editing system comprising the steps of: a) purification of extracellular vesicles (EVs) from red blood cells (RBCs), wherein the RBCs are preferably derived from a human and treated with ionophore in particular calcium ionophore; b) electroporation of the EVs with RNAs which may be Cas9 mRNAs and/or gRNAs to form RNAloaded EVs; and c) applying the RNA-loaded EVs to the target cells. CRISPR is a method that enables robust and precise modifications of genomic DNA for a wide range of applications in research and medicine. The system can be designed to target genomic DNA directly.

In one embodiment, the EVs are electroporated with Cas9 mRNA and gRNA. Preferably, Cas9 mRNA comprises or consists of SEQ ID NO: 2. Further, the gRNA is eGFP gRNA comprising or consisting of SEQ ID NO: 3.

In another embodiment, the EVs are electroporated with Cas9 and gRNA plasmids. In another embodiment, the target cells comprise cancer cells or are cancer cells.

In a further embodiment, the target cells comprise leukemia cells or are leukemia cells. In a particular embodiment, the target cells comprise leukemia cells in particular AML cells, breast cancer cells, or a combination of AML cells and breast cancer cells. In a fourth aspect of the invention, there is provided a method of treating cancer by delivery of RNA to target cells comprising the steps of: a) purification of extracellular vesicles (EVs) from red blood cells (RBCs) which are preferably derived from a mammal in particular a human and treated with ionophore in particular calcium ionophore; b) electroporation of the EVs with RNAs to form RNA-loaded EVs; and c) applying the RNA-loaded EVs to the target cells thereby inhibiting the growth of the target cells, wherein the target cells comprise cancer cells.

In one embodiment, the target cells comprise leukemia cells, breast cancer cells, or a combination of leukemia cells and breast cancer cells. In another embodiment, the target cells comprise acute myeloid leukemia cells.

In another embodiment, the step c) comprises a step of administering the RNA-loaded EVs to a subject having the target cells via a local or systemic administration. Local administration refers to the delivery of the RNA-loaded EVs directly to the site of action, and includes, but not limiting to, intratumoral administration. Systemic administration refers to the delivery of the RNA-loaded EVs via circulatory system, and includes, but not limiting to, intravenous injection.

In a further embodiment, the growth of the target cells is suppressed after the step c).

### SUMMARY OF THE FIGURES

Embodiments and experiments illustrating the principles of the invention will now be discussed with reference to the accompanying figures in which:

FIG. 1. Characterization of EVs from RBCs and uptake of RBCEVs by leukemia cells a) Average concentrations (100,000×dilution) of RBCEVs from three donors±SEM (gray) and their size distribution, determined using a Nanosight nanoparticle analyzer. b) Representative transmission electron microscopy image of RBCEVs. Scale bar: 100 nm. c) Western blot analysis of EV markers ALIX and TSG101; and RBC marker Hemoglobin A (HBA) relative to GAPDH (loading control) in cell lysates and EVs from RBCs. d) Western blot analysis of Stomatin (STOM) and Calnexin (CANX) as the markers of RBCEVs and endoplasmic reticulum, respectively, relative to GAPDH, in leukemia MOLM13 cells, NOMO1 cells, RBCs, and RBCEVs. e) Western blot analysis of HBA relative to GAPDH in leukemia MOLM13 cells untreated or incubated with 8.25×1011 RBCEVs for 24 h. f) Representative immuno fluorescent images of MOLM13-GFP cells incubated with 12.4×1011 PKH26-labeled EVs for 24 h. Scale bar, 20 μm. g) FACS analysis of PKH26 in MOLM13 cells that were incubated with 12.4×1011 unlabeled or PKH26-labeled EVs with and without Heparin for 24 h. The supernatant of the last wash after PKH26 labeling was used to determine the background. Percentages of PKH26-positive cells are indicated above the gates. h) Average percentage of PKH26-positive cells in each condition (mean±SEM, n=3 cell passages). P value (*** P<0.001) was determined using Student's one-tail t-test. In c-e, molecular weights (KDa) of protein markers are shown on the right. Each experiment was repeated two to three times in 2-3 cell passages.

FIG. 2. Electroporation of RBCEVs with ASOs and delivery to leukemia cells. a) Experimental scheme of ASOs delivery by RBCEVs. b) Average concentration of EVs (200× dilution) and average fold change in FAM fluorescent intensity relative to unelectroporated EVs (UE-EVs) of 12 fractions in a sucrose gradient separation of RBCEVs electroporated with a FAM-labeled scrambled negative control ASOs (FAM-NC-ASOs), determined using a Nanosight analyzer and Synergy fluorescent microplate reader, respectively, n=3 repeats. c Separation of unbound NC-ASOs (unlabeled) from 8.25×1011 unelectroporated or electroporated RBCEVs compared to the untreated NC-ASOs (200 pmol) in 10% native gel, visualized using SYBR Gold staining (top) and the average percentage of NC-ASOs unbound or bound to electroporated RBCEVs (bottom), n=3 independent replicates. d) FACS analysis of FAM fluorescence vs. forward scatter area (FSC-A) in MOLM13 cells transfected with 400 pmol FAM-NC-ASO using Lipofectamine™ 3000 (Lipo), INTERFERin® (Inte) or 12.4×1011 RBCEVs. Percentages of FAM-positive cells are indicated above the gate. e) Average percentages of FAM+cells among viable MOLM13 cells transfected or treated with RBCEVs containing FAM ASOs as in d, n=3 repeats. f) Percentages of dead cells determined by propidium iodide staining among MOLM13 cells transfected or treated with RBCEVs containing unlabeled NC ASOs, n=4 repeats. All graphs present mean±SEM. Student's one-tail t-test results are shown as n.s. non-significant; ** P<0.01; *** P<0.001 and **** P<0.0001 relative to the unelectroporated control (c) or to the untreated control (e, f).

FIG. 3. RBCEVs deliver ASOs to leukemia and breast cancer cells for miR-125b inhibition. a) Experimental scheme of ASOs delivery to cancer cells using RBCEVs. b) Percentage of anti-miR-125b ASOs (125b-ASOs) associated with 6.2×1011 unelectroporated or 125b-ASO-electroporated RBCEVs after a treatment with RNase I f for 30 min. c) Copy number of 125b-ASO in MOLM13 cells treated with 12.4×1011 RBCEVs unelectroporated (UE-EVs) or RBCEVs electroporated with NC-ASOs or with 125b-ASOs for 72 h. d) Expression fold change of miR-125b in MOLM13 cells that were incubated with 125b-ASOs alone, 16.8×1011 unelectroporated RBCEVs (UE-EVs), 16.8×1011 NCASOs-loaded RBCEVs, or 4.2 to 16.8×1011 125b-ASOs loaded RBCEVs. miR-125b expression was determined using Taqman qRT-PCR normalized to U6b RNA and presented as average fold change relative to the untreated control. e) Expression fold change of BAK1 in MOLM13 cells treated as in d, determined using SYBR Green qRT-PCR, normalized to GAPDH and presented as average fold change relative to the untreated control. f) Proliferation of MOLM13 cells treated with 12.4×1011 unelectroporated or NC/125b-ASO-electroporated EVs, determined using cell counts. g) Viability of breast cancer CA1a cells (%) treated as in f, determined by crystal violet staining. In all panels, the experiments were repeated three or four times with 3 or 4 cell passages. Bar graphs present mean±SEM. P values were calculated using one-way ANOVA test (d, e) or student's one tail t-test relative to the untreated controls (b, f, g) * P<0.05, ** P<0.01.

FIG. 4. RBCEVs are taken up by breast cancer cells in vivo. a) Schema of an in vivo EV uptake assay. b) Total radiance efficiency of PKH26 fluorescence in the tumors 24 to 72 h after an intratumoral injection of 16.5×1011 PKH26-labeled RBCEVs, determined using an in vivo imaging system (IVIS), presented as mean±SEM (n=3 mice). c) Images of the mice bearing untreated tumors on the right flank and tumors injected with PKH26-labeled EVs on the left flank, 72 h post-treatment, captured using IVIS. PKH26 is shown in pseudocolored radiance. d) Images of the tumors excised from the mice in c. e) Representative confocal microscopy images of tumor sections with DAPI stained nuclei and PKH26 signals from the cells with EV uptake. Scale bar, 20 μm.

FIG. 5. Treatment with ASOs-loaded RBCEVs suppresses tumor growth by miR-125b knockdown. a) Schema of ASOs delivery to nude mice bearing breast cancer xenografts. b) Average bioluminescent photon flux of the tumors treated every 3 days with intratumoral injection of 8.25×1011 RBCEVs containingNC/125b-ASOs (E-EVs, n=8 mice) or with 400 pmol NC/125b-ASOs (n=6 mice), determined using IVIS (mean±SEM). c) Average weight of the mice (mean±SEM). d) Representative images on day 0 and 42. Bioluminescence is shown in pseudocolored radiance. e) Representative pictures of the tumors on day 44. f) Representative H & E staining images of the tumor and the lung collected on day 44. Scale bar, 50 μm. g) miR-125b fold change relative to U6b RNA and NC condition in the tumors after 44 days of treatments, determined using Taqman qRT-PCR (mean±SEM). P values were determined using one-tail Mann—Whitney test b, g: ** P<0.01; *** P<0.001; n.s. non-significant. The whole experiment was performed in three independent repeats (three batches of mice).

FIG. 6. Biodistribution of RBCEVs upon systemic administration in NSG mice. a) Experimental schema for determination of RBCEV circulation time following an i.v. injection. b) FACS analysis of PKH26 fluorescence on the beads that were bound to total EVs from the blood of NSG mice immediately (0 h) or 3, 6, 12 h after the i.v. injection of 3.3×1012 PKH26-labeled RBCEVs. The percentage of PKH26-positive beads are shown above the gate and the average is shown in the bar graph (mean±SEM; n=3 or 4 mice in two repeats). c) Experimental schema for determination of RBCEV biodistribution in NSG mice. d) Representative images of the organs 24 h after 2 i.p. injections (24 h apart) of 3.3×1012 DiR-labeled RBCEVs or the supernatant from the last wash of labeled EVs. Images were captured using IVIS. DiR fluorescence is shown in pseudocolored radiance. e Average DiR radiance in the organs of the mice injected with DiR-labeled RBCEVs (mean±SEM; n=4 mice in 2 repeats). f) Experimental schema for determination of vivotrack-680 (VVT)-labeled RBCEV distribution to the bone marrow in NSG mice. g) FACS analysis of VVT fluorescence (APC-Cy5.5) vs. FSC-A of bone marrow cells from the mice 24 h after 2 i.p. injections (24 h apart) of 3.3×1012 VVT-labeled RBCEVs or the EV wash supernatant (Sup). h) Average percentage of VVT-positive cells (mean±SEM, n=4 mice in 2 repeats). ** P<0.01, one-tail Mann-Whitney test.

FIG. 7. Systemic delivery of miR-125b ASOs in RBCEVs suppresses leukemia progression in AML xenografted mice. a) Experimental schema of AML xenografting and ASOs delivery in NSG mice. b) Average fold change in total body bioluminescence of the mice after 0 to 9 days of treatment with 3.3×1012 RBCEVs containing NC-ASOs (n=7 mice) or 125b-ASOs (n=6 mice) relative to the signals before the treatment started (day 0), determined using an IVIS (mean±SEM). c) Representative images of the leukemic mice on day 0 & 9, captured using the IVIS. Bioluminescence is shown in pseudocolors. d) Average weight of the mice (mean±SEM). e) FACS analysis of GFP cells in the bone marrow of the leukemic mice: representative dot plot of GFP (FITC channel) vs. size scatter area (SSC-A) and the average percentage of GFP-positive cells (mean±SEM, n=3 mice/group). f) Representative H & E staining images of the spleen and liver from a nontransplanted mouse and from AML mice treated with NC/125b-ASOs loaded RBCEVs. Arrows indicate clusters of in filtrating leukemia cells that have larger nuclei than normal cells. Scale bar, 50 μm. g miR-125b expression fold change normalized to U6B RNA in the spleen (n=5 mice) and liver (n=3 mice), determined using Taqman qRT-PCR and presented as mean fold change±SEM relative to NC in the spleen. * P<0.05; ** P<0.01 determined using one-tail Mann-Whitney test (b, e, g). The whole experiment was performed in two independent repeats.

FIG. 8. RBCEVs deliver Cas9 mRNA and gRNA to leukemia cells for genome editing. a) Schema of Cas9 mRNA and gRNA delivery. b Average level of Cas9 mRNA in 6.2×1011 RBCEVs untreated, incubated or electroporated with 6 pmol Cas9 mRNA and treated with RNase If, relative to the unelectroporated Cas9 level (2nd condition). c) The level of Cas9 mRNA relative to GAPDH mRNA in MOLM13 cells that were incubated with 12.4×1011 unelectroporated RBCEVs (UE-EVs) or RBCEVs electroporated with 3, 6, or 12 pmol Cas9 mRNA (E-EVs) after 24 h of treatment, relative to the 3 pmol condition. d) Representative images of MOLM13 cells that were incubated for 48 h with 12.4×1011 UE-EVs or EVs that were electroporated with 6 pmol Cas9 mRNAs. MOLM13 cells were also electroporated directly with 6 pmol Cas9 mRNAs (Cas9 E) for comparison. The cells were stained for HA-Cas9 protein (green) and nuclear DNA (Hoechst, blue). Scale bar, 20 μm. e) Average percentage of MOLM13 cells stained positive for HA-Cas9 protein as shown in d. f) Western blot analysis of Cas9 and α-tubulin (TUB) in MOLM13 cells untreated, treated with 12.4×1011 unelectroporated or 6 pmol-Cas9 mRNA-loaded RBCEVs. Below each band is its mean intensity, quantified using ImageJ. g miR-125b and BAK1 expression fold change, relative to untreated condition, normalized to U6b RNA and 18s RNA respectively, in MOLM13 cells treated with 12.4×1011 UE-EVs or EVs loaded with 6 pmol Cas9 mRNA and mir-125b-targeting gRNA for 48 h. h) Alignment of mir-125b-targeting gRNA with wildtype (×WT) mir-125b (frame indicates mature sequence) and mutant DNA sequences from MOLM13 cells treated as in g. Red, insertion or deletion. Green, mismatch. PAM protospacer adjacent motif. All the bar graphs are presented as mean±SEM (n=3 or 4 repeats of 3 or 4 cell passages). * P<0.05; *** P<0.001; **** P<0.0001: one-tail Student's t-test.

FIG. 9. Purification and characterization of extracellular vesicles (EVs) from human red blood cells (RBCs). a) Purification method: culture supernatants were collected from ionophore-treated human red blood cells and subjected to multiple steps of low speed centrifugation to remove cells and debris. EVs were purified by 3 rounds of ultracentrifugation including one with 60% sucrose cushion at 100,000×g. b) Polydispersity index, and c) Zeta potential of RBCEVs from 3 donors were determined by using a Zetasizer Nano (mean±SEM).

FIG. 10. Morphology and size distribution of RBCEVs after multiple freeze-thaw cycles. a) Representative transmission electron microscopy images of RBCEVs from the same batch after 1-3 freeze-thaw cycles. Images were captured at 42000× (left) and 86000× (right). Scale bar, 200 nm. b) Average concentrations of RBCEVs (100,000× dilution) from three donors±SEM (grey) and their size distribution, determined using a Nanosight analyzer, after 1-3 freeze-thaw cycles.

FIG. 11. RBCEVs are taken up by leukemia MOLM13 cells. a) Schematic presentation of the EV uptake assay: RBCEVs were labeled with PKH26 (a red fluorescent membrane dye), washed three times using ultracentrifugation and incubated with latex beads overnight or with MOLM13 cells for 24 hours. b) FACS analysis of latex beads incubated with 12.4×10 11 unlabeled or PKH26-labeled RBCEVs. The beads were gated based on forward scattering area (FSC-A) and size scattering area (SSC-A). The PKH26 fluorescence (PE channel) was plotted vs. FSC-A. Percentages of PKH26 positive beads are indicated above the gates. c) FACS analysis of MOLM13 cells incubated with 12.4×10 11 unlabeled or PKH26-labeled RBCEVs. The cells were gated based on FSC-A vs. SSC-A (live population) and FSC-width vs. FSC-height (single cells). Percentages of PKH26 positive cells are indicated above the gates.

FIG. 12. Electroporation of RBCEVs with Dextran at different voltages a) Schematic presentation of EV electroporation: 8.25×10 11 RBCEVs were mixed with 4 μg Alexa Fluor® 647 (AF647)-labeled Dextran and electroporated in OptiMEM at different voltages from 50 to 250 V. EVs were incubated with latex beads overnight and analyzed using FACS. b) FACS analysis of AF647 fluorescence (APC channel) and FSC-A of the beads that were incubated with Dextran AF647, electroporated Dextran AF647, electroporated EVs (E-EVs) or unelectroporated EVs (UE-EVs). The percentages of AF647 positive beads are indicated above the gates.

FIG. 13. Characterization of electroporated RBCEVs. a) Schema of top-down sucrose density gradient separation of RBCEVs. b) Concentrations of unelectroporated (UE-EVs) or FAM-ASO-electroporated RBCEVs (E-EVs) in each sucrose fraction (200× dilution) were determined using a Nanosight analyzer and the density of sucrose was determined using a refractometer. c) Size distribution of EVs in fraction 6, determined using the Nanosight particle analyzer.

FIG. 14. Characterization of electroporated RBCEVs. a) FAM fluorescence of the unencapsulated FAM-ASOs from electroporated RBCEVs in 10% native gel. b) Average fluorescent intensity of FAM-ASOs that were incubated with RBCEVs with or without electroporation (E or UE) in OptiMEM containing 50% FBS at 37° C. for 1-72 hours, determined using a Synergy™ microplatereader (mean±SEM). P value was determined using Student's one-tail t-test (n=3 independent repeats). c) Standard curve of 125b-ASOs concentration vs. Ct values were determined using Taqman qRT-PCR.

FIG. 15. RBCEVs deliver Dextran to leukemia MOLM13 cells. a) Schematic presentation of Dextran delivery: 8.25-16.5×10 11 RBCEVs were mixed with 4 μg Dextran AF647 and electroporated at 250 V. Electroporated EVs were incubated with MOLM13 cells for 24 hours. b) FACS analysis of Dextran AF647 fluorescence in MOLM13 cells that were untreated or incubated with 8.25-16.5×10 11 Dextran-AF647 electroporated EVs (E-EVs) or 16.5×10 11 unelectroporated (UE-EVs).

FIG. 16. RBCEVs deliver antisense oligonucleotides (ASOs) to leukemia NOMO1 cells. a) FACS analysis of PKH26 (PE channel) in NOMO1 cells that were untreated or incubated with 12.4×10 11 PKH26-labeled EVs. b) Representative confocal microscopy images of NOMO1 cells treated with PKH26-labeled EVs. Scale bar, 20 μm. c) FACS analysis of FAM fluorescence (FITC channel) in NOMO1 cells that were untreated or incubated with FAM-ASOs or with 12.4×1011 unelectroporated EVs (UE-EVs) or with FAM-ASOs-electroporated EVs (E-EVs).

FIG. 17. Uptake of ASOs by leukemia cells over time. FACS analysis of FAM fluorescence (FITC channel) in MOLM13 cells that were untreated or incubated with 400 pmol FAM-ASOs alone or with 12×1011 FAM-ASOelectroporated RBCEVs for 5 days. The percentages of FAM positive cells are shown above the gates.

FIG. 18. RBCEVs confer higher efficiency and lower toxicity than Lipofectamine™ 3000 and INTERERin® in the delivery of Dextran to MOLM13 cells. a) FACS analysis of AF647 fluorescence in MOLM13 cells that were untreated, incubated with unelectroporated RBCEVs (UE-EVs), with Dextran-AF647 (Dex-647) alone, with Dex-647 loaded Lipofectamin™ 3000 (Lipo3000), with Dex-647 loaded INTERFERin® or with 12.4×1011 Dex-647 electroporated RBC EVs (E-EVs) for 24 hours. b) Percentage of dead cells determined using Propidium iodide (PI) staining in MOLM13 cells treated as in (a). The bar graph represents the average±SEM of 2 to 3 repeats.

FIG. 19. Knockdown of the miR-125 family by EV-delivered ASOs in leukemia and breast cancer cells. a) Expression fold change of miR-125a relative to U6b RNA in MOLM13 cells that were untreated, incubated with 16.8×10 11 unelectroporated RBCEVs (UE-EVs), with 16.8×10 11 NC-ASO electroporated RBCEVs (E-EVs) or 125b-ASOelectroporated RBCEVs at indicated doses for 72 hours. b) Expression fold change of miR-125a and 125b, relative to U6b RNA, in NOMO1 cells treated with indicated doses of 125b-ASO-electroporated RBCEVs. c) Expression fold change of miR-125a and 125b, relative to U6b RNA, in CA1a cells treated with indicated doses of 125b-ASO-electroporated RBCEVs. In all panels, miR-125a, 125b and U6b expression were determined using Taqman qRT-PCR in 3 or 4 cell passages (mean±SEM). One-way Anova test result is shown in each graph.

FIG. 20. Distribution of RBCEVs by systemic administration in nude mice. a) Schematic presentation of the experiment: nude mice with small CA1a tumors (7 mm in diameter) were injected i.p. with 16.5×10 11 PKH26-labeled or DiR-labeled RBCEVs. b) Representative image of the live mice, and c) Representative ex vivo image of the organs from nude mice injected with DiRlabeled RBCEVs or the supernatant of the EV wash at 24 hours post-treatment. DiR fluorescence is presented as pseudocolored radiance (photon/s). d) Cryosections of the organs with PKH26 fluorescence (red) and DAPI staining of the nuclei (blue) from nude mice injected with PKH26-labeled RBCEVs. Scale bar, 10 μm.

FIG. 21. RBCEV treatments do not affect the organs. Representative pictures of tissue sections stained with H & E from untreated and intraperitoneally PKH26-RBCEVs injected mice (as in FIG. 20). Scale bar, 100 μm. The same morphology was observed in other samples (3 mice/group).

FIG. 22. RBCEVs deliver Cas9 mRNA and gRNAs a) Sequences of mir-125 loci in the human genome and the design of gRNA targeting these loci. Sequences were colored by their similarity (black: all identical; blue: half identical) using DNAMAN sequence analysis software. Guide strands are the major strands that are processed into the mature miR-125a or 125b of the miR-125 family. Guide RNA was designed such that mutations may occur in the seed sequence of mature miR-125s (arrow head). b) Expression of miR-125a in MOLM13 cells treated with unelectroporated EVs (UE-EVs) or with EVs that were electroporated with Cas9 mRNA and mir-125b-targeting gRNA for 48 hours (mean±SEM; n=3 cell passages). *P<0.05, student's one-way t-test. c) FACS analysis of GFP in 293T-eGFP cells incubated with UE-EVs or with EVs that were electroporated with Cas9 plasmid and eGFP-targeting gRNA plasmid. GFP negative cells are indicated by the arrow. d) FACS analysis of GFP expression in NOMO1-eGFP cells that were treated with UE-EVs or EVs loaded with Cas9 mRNA and anti-eGFP gRNA for 7 days.

FIG. 23. Supplementary qPCR data with additional internal Controls a) Expression fold change of miR-125b relative to miR-103a in MOLM13 cells that were untreated, incubated with 16.8×10 11 unelectroporated RBC EVs (UE-EVs), with 16.8×1011 NC-ASO electroporated RBC EVs (E-EVs) or 4.2-16.8×1011 125b-ASO-electroporated RBCEVs for 72 hours, determined using miRCURY-LNA qRTPCR (mean±SEM, n=3 cell passages). One-way Anova test result is shown in the graph. b) The level of Cas9 mRNA relative to ACTB and 18S RNA in MOLM13 cells that were incubated with 12.4×1011 unelectroporated RBCEVs (UE-EVs) or 12.4×1011 RBCEVs electroporated with 3, 6 or 12 pmol Cas9 mRNA (E-EVs) after 24 hours of treatment, relative to the 3 pmol condition (mean±SEM; n=3 cell passages).

FIG. 24. Gating strategy for FACS analysis of bone marrow cells FACS analysis of GFP cells in the bone marrow of NSG mice treated with 3.3×1012 RBCEVs containing 125b-ASO as in FIG. 7. Monocytes were gated based on FSC-A and SSC-A to exclude the debris, dead cells and RBCs (low FSC-A). The single cells were further gated from monocytes based on FSC-width vs. FSC-height, to exclude doublets and aggregates. The live cells were gated from the single cells population based on Cytox blue negative (PB450 channel). Subsequently, the GFPpositive cells were gated in FITC channel as the population that exhibit negligible GFP signals in the untreated negative control. The same gates were applied to all samples from the same batch.

FIG. 25. Full images of the native gel electrophoresis a) Separation of unlabeled NC-ASOs (22 bp): 200 pmol unlabeled NC-ASOs (lane 1), 8.25×1011 unelectroporated RBCEVs (lane 2), mixture of 200 pmol NC-ASOs and 8.25×1011 unelectroporated RBCEVs (lane 3), mixture of 200 pmol NC-ASOs and 8.25×1011 RBCEVs after electroporation (lane 4) loaded in 10% native gel and visualized using SYBR Gold staining in a Gel Doc™ EZ Documentation system. b) Separation of FAM-labeled NC-ASOs (22 bp): 200 pmol FAM NC-ASOs (lane 1), 8.25×1011 unelectroporated RBCEVs (lane 2), mixture of 200 pmol FAM NC-ASOs and 8.25×1011 unelectroporated RBCEVs (lane 3), mixture of 200 pmol FAM NCASOs and 8.25×1011 RBCEVs after electroporation (lane 4) loaded in 10% native gel and visualized by FAM fluorescence, using a Gel Doc™ EZ Documentation system.

FIG. 26. Full images of the Western blots a) Western blot analysis of ALIX, TSG101 and HBA relative to GAPDH (loading control) in cell lysates (lane 1) and EVs (lane 2) from RBCs. b) Western blot analysis of HBA and GAPDH in MOLM13 cells untreated (lane 1) or incubated with 8.25×1011 unelectroporated RBCEVs (lane 2) or with 8.25×1011 electroporated RBCEVs (lane 3) for 24 hours. c) Western blot analysis of Stomatin (STOM), Calnexin (CANX), and GAPDH in leukemia MOLM13 cells (lane 1), NOMO1 cells (lane 2), RBCs (lane 3) and RBCEVs (lane 4). d) Western blot analysis of Cas9 and TUB in MOLM13 cells untreated (lane 1), treated with unelectroporated RBCEVs (lane 2) or Cas9 mRNA—loaded RBCEVs (lane 3). In all panels, the blots were cut horizontally and hybridized with multiple antibodies at the same time. Protein ladder (L) was loaded at 2 sides of the samples to determine the molecular weights.

FIG. 27. Distribution of RBCEVs by systemic administration in nude mice Ex vivo image of the organs from NOD scid gamma (NSG) mouse injected i.v. with RBCEVs. Biodistribution by i.v. injection.

FIG. 28. Uptake of Bodipy-labeled RBCEVs by lymphoma B95-8 Cells. a) B95-8 cells +flow-through, and b) B95-8 cells+Bodipy EVs.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Extracellular Vesicles

The term “extracellular vesicle” as used herein refers to a small vesicle-like structure released from a cell into the extracellular environment.

Extracellular vesicles (EVs) are substantially spherical fragments of plasma membrane or endosomal membrane between 50 and 1000 nm in diameter. Extracellular vesicles are released from various cell types under both pathological and physiological conditions. Extracellular vesicles have a membrane. The membrane may be a double layer membrane (i.e. a lipid bilayer). The membrane may originate from the plasma membrane. Accordingly, the membrane of the extracellular vesicle may have a similar composition to the cell from which it is derived. In some aspects disclosed herein, the extracellular vesicles are substantially transparent.

The term extracellular vesicles encompasses exosomes, microvesicles, membrane microparticles, ectosomes, blebs and apoptotic bodies. Extracellular vesicles may be produced via outward budding and fission. The production may be a natural process, or a chemically induced or enhanced process. In some aspects disclosed herein, the extracellular vesicle is a microvesicle produced via chemical induction.

Extracellular vesicles may be classified as exosomes, microvesicles or apoptotic bodies, based on their size and origin of formation. Microvesicles are a particularly preferred class of extracellular vesicle according to the invention disclosed herein. Preferably, the extracellular vesicles of the invention have been shed from the plasma membrane, and do not originate from the endosomal system.

Extracellular vesicles disclosed herein may be derived from various cells, such as red blood cells, white blood cells, cancer cells, stem cells, dendritic cells, macrophages and the like. In a preferred example, the extracellular vesicles are derived from a red blood cell.

Microvesicles or microparticles arise through direct outward budding and fission of the plasma membrane. Microvesicles are typically larger than exosomes, having diameters ranging from 100-500 nm. In some cases, a composition of microvesicles comprises microvesicles with diameters ranging from 50-1000 nm, from 101-1000 nm, from 101-750 nm, from 101-500 nm, or from 100-300 nm, or from 101-300 nm. Preferably, the diameters are from 100-300 nm.

For example, the extracellular vesicle compositions disclosed herein may be substantially uniform in size. They may have a mean diameter of about 100 nm, about 110 nm, about 120 nm, about 130 nm, about 140 nm, about 150 nm, about 160 nm, about 170 nm, about 180 nm, about 190 nm or about 200 nm. In some cases, the mean diameter is about 140 nm and a polydispersity index (PDI) of between 0.05 and 0.09, between 0.06 and 0.08, or around 0.07.

Exosomes range from around 30 to around 100 nm. They are observed in a variety of cultured cells including lymphocytes, dendritic cells, cytotoxic T cells, mast cells, neurons, oligodendrocytes, Schwann cells, and intestinal epithelial cells. Exosomes originate from the endosomal network that locates in within mutivesicular bodies, large sacs in the cytoplasm. These sacs fuse to the plasma membrane, before being released into extracellular environment.

Apoptotic bodies or blebs are the largest extracellular vesicle, ranging from 1-5 μm. Nucleated cells undergoing apoptosis pass through several stages, beginning with condensation of the nuclear chromatin, membrane blebbing and finally release of EVs including apoptotic bodies.

Preferably, the extracellular vesicles are derived from human cells, or cells of human origin. The extracellular vesicles of the invention may have been induced from cells contacted with a vesicle inducing agent. The vesicle inducing agent may be calcium ionophore, lysophosphatidic acid (LPA), or phorbol-12-myristat-13-acetate (PMA).

### Red Blood Cell Extracellular Vesicles (RBC-EVs)

In certain aspects disclosed herein, the extracellular vesicles are derived from red blood cells. Red blood cells are a good source of EVs for a number of reasons. Because red blood cells are enucleated, RBC-EVs contain less nucleic acid than EVs from other sources. RBC-EVs do not contain endogenous DNA. RBC-EVs may contain miRNA or other RNAs. RBC-EVs are free from oncogenic substances such as oncogenic DNA or DNA mutations.

RBC-EVs may comprise haemoglobin and/or stomatin and/or flotillin-2. They may be red in colour. Typically RBC-EVs exhibit a domed (concave) surface, or “cup shape” under transmission electron microscopes. The RBC-EV may be characterised by having cell surface CD235a. RBC-EVs according to the invention may be about 100 to about 300 nm in diameter. In some cases, a composition of RBC-EVs comprises RBC-EVs with diameters ranging from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the diameters are from 100-300 nm. A population of RBC-EVs will comprise RBC-EVs with a range of different diameters, the median diameter of RBC-EVs within a RBC-EV sample can range from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the median diameter is between 100-300 nm.

Preferably, the RBC-EVs are derived from a human or animal blood sample or red blood cells derived from primary cells or immobilized red blood cell lines. The blood cells may be type matched to the patient to be treated, and thus the blood cells may be Group A, Group B, Group AB, Group O or Blood Group Oh. Preferably the blood is Group O. The blood may be rhesus positive or rhesus negative. In some cases, the blood is Group O and/or rhesus negative, such as Type O−. The blood may have been determined to be free from disease or disorder, such as free from HIV, sickle cell anaemia, malaria. However, any blood type may be used. In some cases, the RBC-EVs are autologous and derived from a blood sample obtained from the patient to be treated. In some cases, the RBC-EVs are allogenic and not derived from a blood sample obtained from the patient to be treated.

RBC-EVs may be isolated from a sample of red blood cells. Protocols for obtaining EVs from red blood cells are known in the art, for example in Danesh et al. (2014) Blood. 2014 Jan 30; 123(5): 687-696. Methods useful for obtaining EVs may include the step of providing or obtaining a sample comprising red blood cells, inducing the red blood cells to produce extracellular vesicles, and isolating the extracellular vesicles. The sample may be a whole blood sample. Preferably, cells other than red blood cells have been removed from the sample, such that the cellular component of the sample is red blood cells. Preferably, the red blood cell sample is completely or substantially leukocyte free.

The red blood cells in the sample may be concentrated, or partitioned from other components of a whole blood sample, such as white blood cells. Red blood cells may be concentrated by centrifugation. The sample may be subjected to leukocyte reduction.

The sample comprising red blood cells may comprise substantially only red blood cells. Extracellular vesicles may be induced from the red blood cells by contacting the red blood cells with a vesicle inducing agent. The vesicle inducing agent may be calcium ionophore, lysophosphatidic acid (LPA), or phorbol-12-myristat-13-acetate (PMA).

RBC-EVs may be isolated by centrifugation (with or without ultracentrifugation), precipitation, filtration processes such as tangential flow filtration, or size exclusion chromatography. In this way, RBC-EVs may be separated from RBCs and other components of the mixture.

Extracellular vesicles may be obtained from red blood cells by a method comprising: obtaining a sample of red blood cells; contacting the red blood cells with a vesicle inducing agent; and isolating the induced extracellular vesicles.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells. The red blood cell sample may additionally comprise plasma, serum, buffer or another carrier. The red blood cells may be diluted in buffer such as PBS prior to contacting with the vesicle inducing agent. The vesicle inducing agent may be calcium ionophore, lysophosphatidic acid (LPA) or phorbol-12-myristat-13-acetate (PMA). The vesicle inducing agent may be about 10 nM calcium ionophore. The red blood cells may be contacted with the vesicle inducing agent overnight, or for at least 1, at least 2, at least 3, at least 4, at least 5, at least 6, at least 7, at least 8, at least 9, at least 10, at least 11, at least 12 or more than 12 hours. The mixture may be subjected to low speed centrifugation to remove RBCs, cell debris, or other non-RBC-EVs matter and/or passing the supernatant through an about 0.45 um syringe filter. RBC-EVs may be concentrated by ultracentrifugation, such as centrifugation at around 100,000×g. The RBC-EVs may be concentrated by ultracentrifugation for at least 10 minutes, at least 20 minutes, at least 30 minutes, at least 40 minutes, at least 50 minutes or at least one hour. The concentrated RBC-EVs may be suspended in cold PBS. They may be layered on a 60% sucrose cushion. The sucrose cushion may comprise frozen 60% sucrose. The RBC-EVs layered on the sucrose cushion may be subject to ultracentrugation at 100,000×g for at least one hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or more. Preferably, the RBC-EVs layered on the sucrose cushion may be subject to ultracentrugation at 100,000×g for about 16 hours. The red layer above the sucrose cushion is then collected, thereby obtaining RBC-EVs. The obtained RBC-EVs may be subject to further processing, such as washing, and optionally loading.

### Purification of Extracellular Vesicles From Red Blood Cells

In certain aspects disclosed herein, the extracellular vesicles are derived from red blood cells. Red blood cells are a good source of EVs for a number of reasons. Because red blood cells are enucleated, RBC-EVs contain less nucleic acid than EVs from other sources. RBC-EVs do not contain endogenous DNA.

RBC-EVs may contain miRNA or other RNAs. RBC-EVs are free from oncogenic substances such as oncogenic DNA or DNA mutations.

RBC-EVs may comprise haemoglobin and/or stomatin and/or flotillin-2. They may be red in colour. Typically RBC-EVs exhibit a domed (concave) surface, or “cup shape” under transmission electron microscopes. The RBC-EV may be characterised by having cell surface CD235a. RBC-EVs according to the invention may be about 100 to about 300 nm in diameter. In some cases, a composition of RBC-EVs comprises RBC-EVs with diameters ranging from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the diameters are from 100-300 nm. A population of RBC-EVs will comprise RBC-EVs with a range of different diameters, the median diameter of RBC-EVs within a RBC-EV sample can range from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the median diameter is between 100-300 nm.

Preferably, the RBC-EVs are derived from a human or animal blood sample or red blood cells derived from primary cells or immobilized red blood cell lines. The blood cells may be type matched to the patient to be treated, and thus the blood cells may be Group A, Group B, Group AB, Group O or Blood Group Oh. Preferably the blood is Group O. The blood may be rhesus positive or rhesus negative. In some cases, the blood is Group O and/or rhesus negative, such as Type O−. The blood may have been determined to be free from disease or disorder, such as free from HIV, sickle cell anaemia, malaria. However, any blood type may be used. In some cases, the RBC-EVs are autologous and derived from a blood sample obtained from the patient to be treated. In some cases, the RBC-EVs are allogenic and not derived from a blood sample obtained from the patient to be treated.

RBC-EVs may be isolated from a sample of red blood cells. Protocols for obtaining EVs from red blood cells are known in the art, for example in Danesh et al. (2014) Blood. 2014 Jan 30; 123(5): 687-696. Methods useful for obtaining EVs may include the step of providing or obtaining a sample comprising red blood cells, inducing the red blood cells to produce extracellular vesicles, and isolating the extracellular vesicles. The sample may be a whole blood sample. Preferably, cells other than red blood cells have been removed from the sample, such that the cellular component of the sample is red blood cells.

The red blood cells in the sample may be concentrated, or partitioned from other components of a whole blood sample, such as white blood cells. Red blood cells may be concentrated by centrifugation. The sample may be subjected to leukocyte reduction.

The sample comprising red blood cells may comprise substantially only red blood cells. Extracellular vesicles may be induced from the red blood cells by contacting the red blood cells with a vesicle inducing agent. The vesicle inducing agent may be calcium ionophore, lysophosphatidic acid (LPA), or phorbol-12-myristat-13-acetate (PMA).

RBC-EVs may be isolated by centrifugation (with or without ultracentrifugation), precipitation, filtration processes such as tangential flow filtration, or size exclusion chromatography. In this way, RBC-EVs may be separated from RBCs and other components of the mixture.

Extracellular vesicles may be obtained from red blood cells by a method comprising: obtaining a sample of red blood cells; contacting the red blood cells with a vesicle inducing agent; and isolating the induced extracellular vesicles.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells. The red blood cells may be diluted in buffer such as PBS prior to contacting with the vesicle inducing agent. The vesicle inducing agent may be calcium ionophore, lysophosphatidic acid (LPA) or phorbol-12-myristat-13-acetate (PMA). The vesicle inducing agent may be about 10 nM calcium ionophore. The red blood cells may be contacted with the vesicle inducing agent overnight, or for at least 1, at least 2, at least 3, at least 4, at least 5, at least 6, at least 7, at least 8, at least 9, at least 10, at least 11, at least 12 or more than 12 hours. The mixture may be subjected to low speed centrifugation to remove RBCs, cell debris, or other non-RBC-EVs matter and/or passing the supernatant through an about 0.45 um syringe filter. RBC-EVs may be concentrated by ultracentrifugation, such as centrifugation at around 100,000×g. The RBC-EVs may be concentrated by ultracentrifugation for at least 10 minutes, at least 20 minutes, at least 30 minutes, at least 40 minutes, at least 50 minutes or at least one hour. The concentrated RBC-EVs may be suspended in cold PBS. They may be layered on a 60% sucrose cushion. The sucrose cushion may comprise frozen 60% sucrose. The RBC-EVs layered on the sucrose cushion may be subject to ultracentrugation at 100,000×g for at least one hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or more. Preferably, the RBC-EVs layered on the sucrose cushion may be subject to ultracentrugation at 100,000×g for about 16 hours. The red layer above the sucrose cushion is then collected, thereby obtaining RBC-EVs. The obtained RBC-EVs may be subject to further processing, such as washing, tagging, and optionally loading.

### Carqo

Extracellular vesicles disclosed herein may be loaded with, or contain, a cargo. The cargo, also referred to as the load, may be a nucleic acid, peptide, protein, small molecule, sugar or lipid. The cargo may be a non-naturally occurring or synthetic molecule. The cargo may be a therapeutic molecule, such as a therapeutic oligonucleotide, peptide, small molecule, sugar or lipid. In some cases, the cargo is not a therapeutic molecule, for example a detectable moiety or visualization agent. The cargo may exert a therapeutic effect in the target cell after being delivered to that target cell. For example, the cargo may be a nucleic acid which is expressed in the target cell. It may act to inhibit or enhance the expression of a particular gene or protein of interest. For example, the protein or nucleic acid may be used to edit a target gene for gene silencing or modification.

Preferably, the cargo is an exogenous molecule, sometimes referred to as a “non-endogenous substance”. In other words, the cargo is a molecule that does not naturally occur in the extracellular vesicle, or the cell from which it is derived. Such a cargo is preferably loaded into the extracellular vesicles after the vesicles have formed, rather than loaded or produced by the cell, such that it is also contained within the extracellular vesicles.

In some cases, the cargo may be a nucleic acid. The cargo may be RNA or DNA. The nucleic acid may be single stranded or double stranded. The cargo may be an RNA. The RNA may be a therapeutic RNA. The RNA may be a small interfering RNA (siRNA), a messenger RNA (mRNA), a guide RNA (gRNA), a circular RNA, a microRNA (miRNA), a piwiRNA (piRNA), a transfer RNA (tRNA), or a long noncoding RNA (IncRNA) produced by chemical synthesis or in vitro transcription. In some cases, the cargo is an antisense oligonucleotide, for example, having a sequence that is complementary to an endogenous nucleic acid sequence such as a transcription factor, miRNA or other endogenous mRNA.

The cargo may be encode a molecule of interest. For example, the cargo may be an mRNA that encodes Cas9 or another nuclease.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (see e.g. Marcus-Sakura, Anal. Biochem. 1988, 172:289). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors. Antisense nucleic acid molecules may stimulate RNA interference (RNAi).

Thus, an antisense nucleic acid cargo may interfere with transcription of target genes, interfere with translation of target mRNA and/or promote degradation of target mRNA. In some cases, an antisense nucleic acid is capable of inducing a reduction in expression of the target gene.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

RNAi and siRNA are described in, for example, Dana et al., Int J Biomed Sci. 2017; 13(2): 48-57, herein incorporated by reference in its entirety. An antisense nucleic acid molecule may contain double-stranded RNA (dsRNA) or partially double-stranded RNA that is complementary to a target nucleic acid sequence, for example FHR-4. A double-stranded RNA molecule is formed by the complementary pairing between a first RNA portion and a second RNA portion within the molecule. The length of an RNA sequence (i.e. one portion) is generally less than 30 nucleotides in length (e.g. 29, 28, 27, 26, 25, 24, 23, 22, 21, 20, 19, 18, 17, 16, 15, 14, 13, 12, 11, 10 or fewer nucleotides). In some embodiments, the length of an RNA sequence is 18 to 24 nucleotides in length. In some siRNA molecules, the complementary first and second portions of the RNA molecule form the “stem” of a hairpin structure. The two portions can be joined by a linking sequence, which may form the “loop” in the hairpin structure. The linking sequence may vary in length and may be, for example, 5, 6, 7, 8, 9, 10, 11, 12, or 13 nucleotides in length. Suitable linking sequences are known in the art.

Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

The nucleic acid molecule may be a miRNA. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length)In some cases, the nucleic acid is synthetic or recombinant.

The nucleic acid disclosed herein may comprise one or more modifications, or non-naturally occurring elements or nucleic acids. In preferred aspects, the nucleic acid comprises a 2′-O-methyl analog. In some cases, the nucleic acid includes a 3′ phosphorothioate internucleotide linkage or other locked nucleic acid (LNA). In some cases, the nucleic acid comprises an ARCA cap. Other chemically modified nucleic acids or nucleotides may be used, for example, 2′-position sugar modifications, 2′-O-methylation, 2′-Fluoro modifications, 2′NH₂ modifications, 5-position pyrimidine modifications, 8-position purine modifications, modifications at exocyclic amines, substitution of 4-thiouridine, substitution of 5-bromo, or 5-iodo-uracil, backbone modifications, methylations, unusual base-pairing combinations such as isocytidine and isoguanidine and the like. Modifications can also include 3′ and 5′ modifications such as capping. For example, the nucleic acid may be PEGylated.

Nucleic acids useful in the methods of the invention include antisense oligonucleotides, mRNA, siRNAs or gRNAs that target oncogenic miRNAs (also known as oncomiRs) or transcription factors. The cargo may be a ribozyme or aptamer. In some cases, the nucleic acid is a plasmid.

The nucleic acid molecule may be an aptamer. The term “aptamer” as used herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington AD, Szostak JW, Nature 1990, 346:818-822; Tuerk C, Gold L. Science 1990, 249:505-510) or by developing SOMAmers (slow off-rate modified aptamers) (Gold L et al. (2010) Aptamer-based multiplexed proteomic technology for biomarker discovery. PLoS ONE 5(12):e15004).

In certain aspects described herein, the cargo is an antisense oligonucleotide (ASO). The antisense oligonucleotide may be complementary to a miRNA or mRNA. The antisense oligonucleotide comprises at least a portion which is complementary in sequence to a target mRNA sequence. The antisense oligonucleotide may bind to, and thereby inhibit, the target sequence. For example, the antisense oligonucleotide may inhibit the translation process of the target sequence. The miRNA may be a miRNA associated with cancer (Oncomir). The miRNA may be miR-125b. The ASO may comprise or consist of the sequence 5′-UCACAAGUUAGGGUCUCAGGGA-3′.

In some aspects, the cargo is one or more components of a gene editing system. For example, a CRISPR/Cas9 gene editing system. For example, the cargo may include a nucleic acid which recognises a particular target sequence. The cargo may be a gRNA. Such gRNAs may be useful in CRISPR/Cas9 gene editing. The cargo may be a Cas9 mRNA or a plasmid encoding Cas9. Other gene editing molecules may be used as cargo, such as zinc finger nucleases (ZFNs) or Transcription activator-like effector nucleases (TALENs). The cargo may comprise a sequence engineered to target a particular nucleic acid sequence in a target cell. The gene editing molecule may specifically target a miRNA. For example, the gene editing molecule may be a gRNA that targets miR-125b. the gRNA may comprise or consist of the sequence 5′-CCUCACAAGUUAGGGUCUCA-3′.

In some embodiments the methods employ target gene editing using site-specific nucleases (SSNs). Gene editing using SSNs is reviewed e.g. in Eid and Mahfouz, Exp Mol Med. 2016 Oct; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include ZFNs, TALENs and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a Fokl endonuclease domain). The DNA-binding domain may be identified by screening a Zince Finger array capable of binding to the target nucleic acid sequence.

TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a Fokl endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326(5959):1501.).

CRISPR is an abbreviation of Clustered Regularly Interspaced Short Palindromic Repeats. The term was first used at a time when the origin and function of these sequences were not known and they were assumed to be prokaryotic in origin. CRISPR are segments of DNA containing short, repetitive base sequences in a palindromic repeat (the sequence of nucleotides is the same in both directions). Each repetition is followed by short segments of spacer DNA from previous integration of foreign DNA from a virus or plasmid. Small clusters of CAS (CRISPR-associated) genes are located next to CRISPR sequences. RNA harboring the spacer sequence helps Cas (CRISPR-associated) proteins recognize and cut foreign pathogenic DNA. Other RNA-guided Cas proteins cut foreign RNA. A simple version of the CRISPR/Cas system, CRISPRiCas9, has been modified to edit genomes. By delivering the Cas9 nuclease and a synthetic guide RNA (gRNA) into a cell, the cell×'s genome can be cut at a desired location, allowing existing genes to be removed and/or new ones added. CRISPR-Cas systems fall into two classes. Class I systems use a complex of multiple Cas proteins to degrade foreign nucleic acids. Class 2 systems use a single large Cas protein for the same purpose. Class 1 is divided into types I, III, and IV; class 2 is divided into types H, V, and V×i CRISPR genome editing uses a type II CRISPR system.

In some aspects, the EV is loaded with a CRISPR related cargo. In other words, the EV is useful in a method involving gene editing, such as therapeutic gene editing. In some cases, the EV is useful for in vitro gene editing.

The cargo may be a guide RNA. The guide RNA may comprise a CRIPSR RNA (crRNA) and a trans-activating CRISPR RNA (tracrRNA). The crRNA contains a guide RNA that locates the correct section of host DNA along with a region that binds to tracrRNA forming an active complex. The tracrRNA binds to crRNA and forms the active complex. The gRNA combines both the tracrRNA and a crRNA, thereby encoding an active complex. The gRNA may comprise multiple crRNAs and tracrRNAs. The gRNA may be designed to bind to a sequence or gene of interest. The gRNA may target a gene for cleavage. Optionally, an optional section of DNA repair template is included. The repair template may be utilized in either non-homologous end joining (NHEJ) or homology directed repair (HDR).

The cargo may be a nuclease, such as a Cas9 nuclease. The nuclease is a protein whose active form is able to modify DNA. Nuclease variants are capable of single strand nicking, double strand break, DNA binding or other different functions. The nuclease recognises a DNA site, allowing for site specific DNA editing.

The gRNA and nuclease may be encoded on a plasmid. In other words, the EV cargo may comprise a plasmid that encodes both the gRNA and the nuclease. In some cases, an EV contains the gRNA and another EV contains or encodes the nuclease. In some cases, an EV contains a plasmid encoding the gRNA, and a plasmid encoding the nuclease. Thus, in some aspects, a composition is provided comprising EVs, wherein a portion of the EVs comprise or encode the nuclease such as Cas9, and a portion of the EVs comprise or encode the gRNA. In some cases, a composition containing EVs that comprise or encode the gRNA and a composition containing EVs that encode or contain the nuclease are co-administered. In some cases, the composition comprises EVS wherein the EVs contain an oligonucleotide that encodes both a gRNA and a nuclease.

CRISPR/Cas9 and related systems e.g. CRISPR×/Cpf1, CRISPR×/C2c1, CRISPR×/C2c2 and CRISPR×/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some cases, the nucleic acid encodes or targets one or more dedifferentiation factors, such as one or more nucleic acids encoding the “Yamanaka factors”, Oct4, Sox2, Klf4 and Myc.

In some cases, the cargo is a peptide or protein. It may be a recombinant peptide or protein. Suitable peptides or proteins include enzymes, such as gene editing enzymes such as Cas9, a ZFN, or a TALEN.

Suitable small molecules include cytotoxic reagents and kinase inhibitors. The small molecule may comprise a fluorescent probe and/or a metal. For example, the cargo may comprise a superparamagnetic particle such as an iron oxide particle. The cargo may be an ultra-small superparamagnetic iron oxide particle such as an iron oxide nanoparticle.

In some cases, the cargo is a detectable moiety such as a fluorescent dextran. The cargo may be radioactively labelled.

Cargo may be loaded into the extracellular vesicles by electroporation. Electroporation, or electropermeabilization, is a microbiology technique in which an electrical field is applied to cells in order to increase the permeability of the cell membrane, allowing chemicals, drugs, or DNA to be introduced into the cell. In other words, the extracellular vesicles may be induced or force to encapsulate the cargo by electroporation. As such, methods disclosed herein may involve a step of electroporating an extracellular vesicle in the presence of a cargo molecule, or electroporating a mixture of extracellular vesicles and cargo molecules.

In other methods disclosed herein, cargo is loaded into the extracellular vesicles by sonication, ultrasound, lipofection or hypotonic dialysis.

### Loading Methodology

Methods suitable for loading cargo into the extracellular vesicles may require a temporary or semi-permanent increase in the permeability of the membrane of the extracellular vesicle. Suitable methods include electroporation, sonication, ultrasound, lipofection or hypotonic dialysis. In preferred methods disclosed herein, RBC-EVs are contacted with a cargo to form a mixture, and the mixture is treated to increase the permeability of the membrane of the extracellular vesicles. The mixture may be chilled prior to treatment. It may further involve one or more buffers, such as PBS.

In a preferred method, cargo is loaded into the extracellular vesicles by electroporation. Electroporation, or electropermeabilization, is a microbiology technique in which an electrical field is applied to cells in order to increase the permeability of the cell membrane, allowing chemicals, drugs, or DNA to be introduced into the cell. In other words, the extracellular vesicles may be induced or forced to encapsulate the cargo by electroporation. Electroporation works by passing thousands of volts across a distance of one to two millimeters of suspended cells in an electroporation cuvette (1.0-1.5 kV, 250-750V/cm). Generally, electroporation is a multi-step process, with several distinct phase. First, a short electrical pulse is applied. Typical parameters would be 300-400 mV for <1 ms across the membrane. Upon application of this potential the membrane charges like a capacitor through the migration of ions from the surrounding solution. Once the critical field is achieved there is a rapid localized rearrangement in lipid morphology, The resulting structure is believed to be a “pre-pore” since it is not electrically conductive but leads rapidly to the creation of a conductive pore. Evidence for the existence of such pre-pores comes mostly from the “flickering” of pores, which suggests a transition between conductive and insulating states. It has been suggested that these pre-pores are small (˜3 A) hydrophobic defects. If this theory is correct, then the transition to a conductive state could be explained by a rearrangement at the pore edge, in which the lipid heads fold over to create a hydrophilic interface. Finally, these conductive pores can either heal, resealing the bilayer or expand, eventually rupturing it. The resultant fate depends on whether the critical defect size was exceeded which in turn depends on the applied field, local mechanical stress and bilayer edge energy. The success of in vivo electroporation depends greatly on voltage, repetition, pulses, and duration. Methods disclosed herein may involve subjecting red blood cell derived extracellular vesicles to electroporation at between about 25 and 300 V, or between about 50 and 250V.

Alternatively, cargo may be loaded into the extracellular vesicles by sonication. Sonication is the act of applying sound energy to agitate particles in a sample, for various purposes such as the extraction of multiple compounds from plants, microalgae and seaweeds. Ultrasonic frequencies (>20 kHz) are usually used, leading to the process also being known as ultrasonication or ultra-sonication. Sonication may be applied using an ultrasonic bath or an ultrasonic probe, colloquially known as a sonicator.

In another method, cargo is loaded with ultrasound. Ultrasound has been shown to disrupt cell membranes, and thereby load cells with molecules. Sound waves with frequencies from 20 kHz up to several gigahertz may be applied to the RBC-EVs.

In yet another method, cargo may be loaded into RBC-EVs by lipofection. Lipofection (or liposome transfection) is a technique used to inject genetic material into a cell by means of liposomes, which are vesicles that can easily merge with the cell membrane since they are both made of a phospholipid bilayer.

### Compositions

Disclosed herein are compositions comprising extracellular vesicles.

The compositions may comprise between 10⁶ to 10¹⁴ particles per ml. The compositions may comprise at least 10⁵ particles per ml, at least 10⁶ particles per ml, at least at least 10^{×7} particles per ml, at least 10⁸ particles per ml, at least 10⁹ particles per ml, at least 10¹⁰ particles per ml, at least 10¹¹ particles per ml, at least 10¹² particles per ml, at least 10¹³ particles per ml or at least 10¹⁴ particles per ml.

The composition may comprise extracellular vesicles have substantially homologous dimensions. For example, the extracellular vesicles may have diameters ranging from 100-500 nm. In some cases, a composition of microvesicles comprises microvesicles with diameters ranging from 50-1000 nm, from 101-1000 nm, from 101-750 nm, from 101-500 nm, or from 100-300 nm, or from 101-300 nm. Preferably, the diameters are from 100-300 nm. In some compositions, the mean diameter of the microvesicles is 100-300 nm, preferably 150-250 nm, preferably about 200 nm.

In some compositions, the extracellular vesicles contain a cargo. Although it is desirable in such compositions for the cargo to be encapsulated into substantially all of the extracellular vesicles in a composition, compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, or at least 97% of the extracellular vesicles contain the cargo. Preferably, at least 85%, at least 90%, at least 95%, or at least 97% of the extracellular vesicles contain the cargo. In some cases, different extracellular vesicles within the composition contain different cargo. In some cases, the extracellular vesicles contain the same, or substantially the same, cargo molecule.

The composition may be a pharmaceutical composition. The composition may comprise one or more extracellular vesicle, and optionally a pharmaceutically acceptable carrier. Pharmaceutical compositions may be formulated for administration by a particular route of administration. For example, the pharmaceutical composition may be formulated for intravenous, intratumoral, intraperitoneal, intradermal, subcutaneous, intranasal or other administration route.

Compositions may comprise a buffer solution. Compositions may comprise a preservative compound. Compositions may comprise a pharmaceutically acceptable carrier.

### Methods of Treatment and Uses of Extracellular Vesicles

Extracellular vesicles disclosed herein are useful in methods of treatment. In particular, the methods are useful for treating a subject suffering from a disorder associated with a target gene, the method comprising the step of administering an effective amount of a modified extracellular vesicle to said subject, wherein the modified extracellular vesicle comprises a binding molecule on its surface and encapsulates a non-endogenous substance for interacting with the target gene in a target cell. The non-endogenous substance may be a nucleic acid for said treatment.

The extracellular vesicles disclosed herein are particularly useful for the treatment of a genetic disorder, inflammatory disease, cancer, autoimmune disorder, cardiovascular disease or a gastrointestinal disease.

In some cases, the disorder is a genetic disorder selected from thalassemia, sickle cell anemia, or genetic metabolic disorder. In some cases, the extracellular vesicles are useful for treating a disorder of the liver, bone marrow, lung, spleen, brain, pancreas, stomach or intestine.

In certain aspects, the extracellular vesicles are useful for the treatment of cancer. Extracellular vesicles disclosed herein may be useful for inhibiting the growth or proliferation of cancerous cells. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine.

The target cell depends on the disorder to be treated. For example, the target cell may be a breast cancer cell, a colorectal cancer cell, a lung cancer cell, a kidney cancer cell or the like. The cargo may be a nucleic acid for inhibiting or enhancing the expression of the target gene, or performing gene editing to silence the particular gene.

Extracellular vesicles and compositions described herein may be administered, or formulated for administration, by a number of routes, including but not limited to systemic, intratumoral, intraperitoneal, parenteral, intravenous, intra-arterial, intradermal, subcutaneous, intramuscular, oral and nasal. Preferably, the extracellular vesicles are administered by a route selected from intratumoral, intraperitoneal or intravenous. The medicaments and compositions may be formulated in fluid or solid form. Fluid formulations may be formulated for administration by injection to a selected region of the human or animal body.

The extracellular vesicle may comprise a therapeutic cargo. The therapeutic cargo may be a non-endogenous substance for interacting with a target gene in a target cell.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Extracellular vesicles may be administered alone or in combination with other treatments, either simultaneously or sequentially dependent upon the condition to be treated.

Extracellular vesicles loaded with a cargo as described herein may be used to deliver that cargo to a target cell. In some cases, the method is an in vitro method. In particularly preferred in vitro methods the cargo is a labelling molecule or a plasmid.

The subject to be treated may be any animal or human. The subject is preferably mammalian, more preferably human. The subject may be a non-human mammal, but is more preferably human. The subject may be male or female. The subject may be a patient. Therapeutic uses may be in humans or animals (veterinary use).

### Protein Expression

Molecular biology techniques suitable for the producing peptides such as peptide, polypeptide or protein cargo according to the invention in cells are well known in the art, such as those set out in Sambrook et al., Molecular Cloning: A Laboratory Manual, New York: Cold Spring Harbor Press, 1989.

The peptide may be expressed from a nucleotide sequence. The nucleotide sequence may be contained in a vector present in the cell, or may be incorporated into the genome of the cell.

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express plant aspartic proteases from a vector according to the invention. Suitable vectors include plasmids, binary vectors, viral vectors and artificial chromosomes (e.g. yeast artificial chromosomes).

In this specification the term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Any cell suitable for the expression of polypeptides may be used for producing peptides according to the invention. The cell may be a prokaryote or eukaryote. Preferably the cell is a eukaryotic cell such as a yeast cell, a plant cell, insect cell or a mammalian cell. In some cases the cell is not a prokaryotic cell because some prokaryotic cells do not allow for the same post-translational modifications as eukaryotes.

In addition, very high expression levels are possible in eukaryotes and proteins can be easier to purify from eukaryotes using appropriate tags. Specific plasmids may also be utilised which enhance secretion of the protein into the media.

Methods of producing a peptide of interest may involve culture or fermentation of a eukaryotic cell modified to express the peptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted aspartic protease. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express peptide of interest, that peptide is preferably isolated. Any suitable method for separating proteins from cell culture known in the art may be used. In order to isolate a protein of interest from a culture, it may be necessary to first separate the cultured cells from media containing the protein of interest. If the protein of interest is secreted from the cells, the cells may be separated from the culture media that contains the secreted protein by centrifugation. If the protein of interest collects within the cell, for example in the vacuole of the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the protein of interest.

It may then be desirable to isolate the protein of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Once the protein of interest has been isolated from culture it may be necessary to concentrate the protein. A number of methods for concentrating a protein of interest are known in the art, such as ultrafiltration or lyophilisation

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

### RNA Druq Delivery Using Red Blood Cell Extracellular Vesicles and Methods Thereof

### Purification and Characterization of RBCEVs

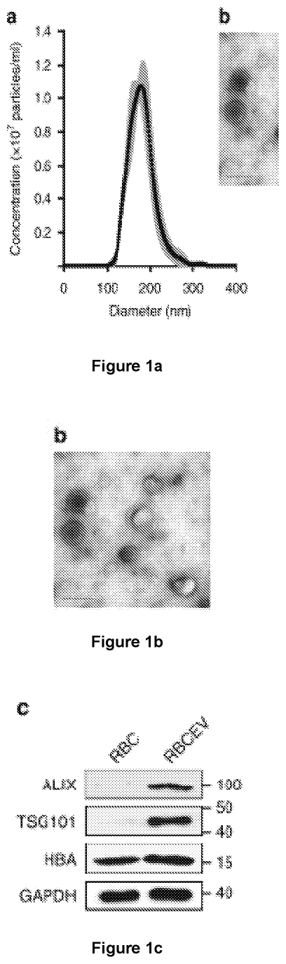

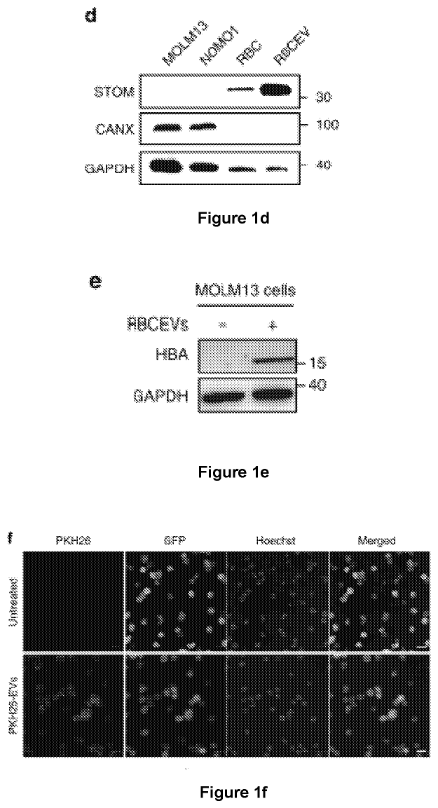

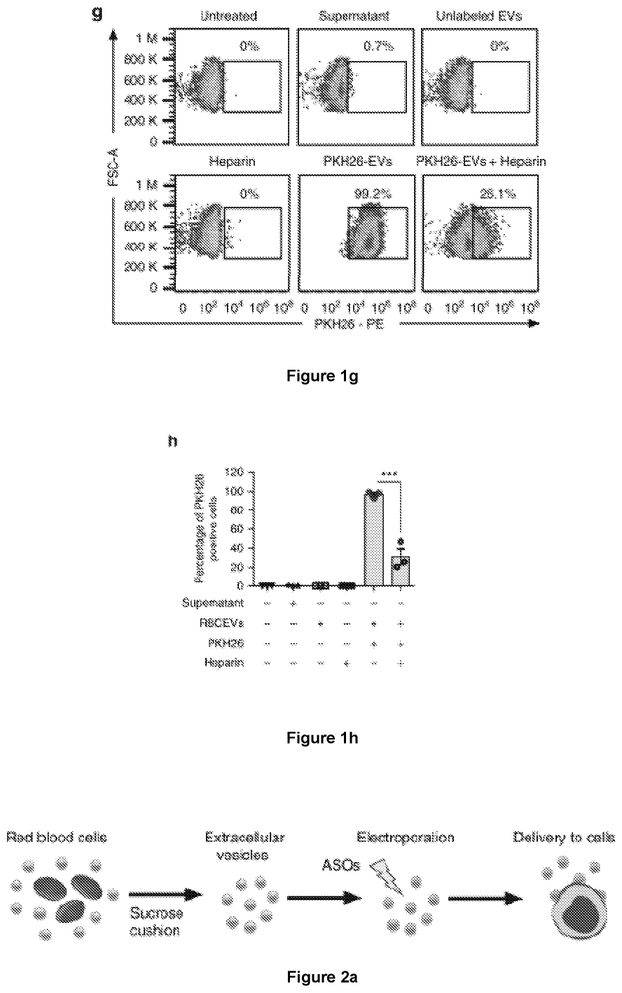

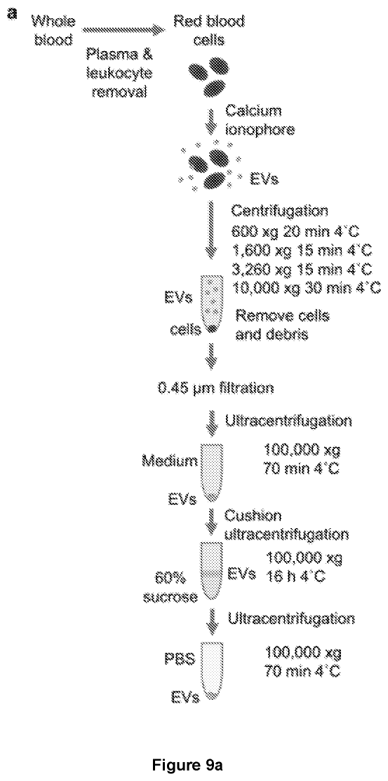

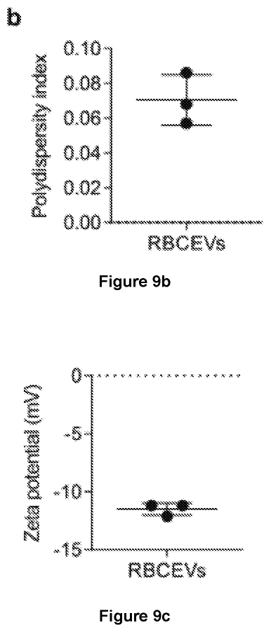

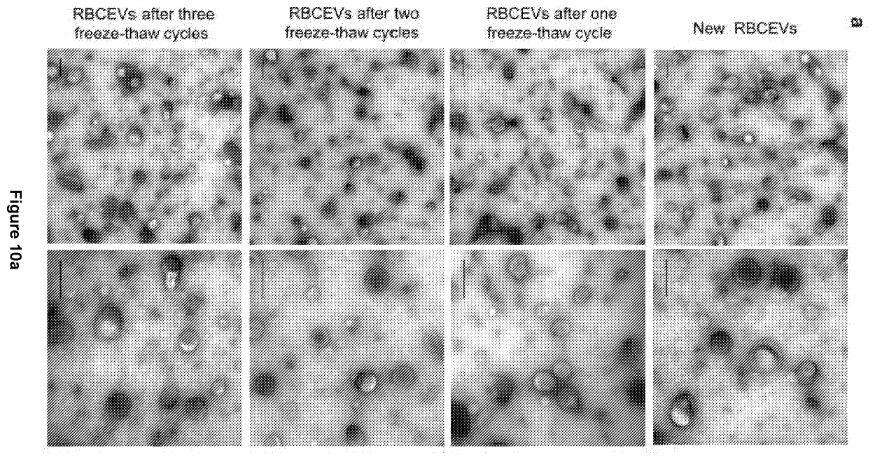

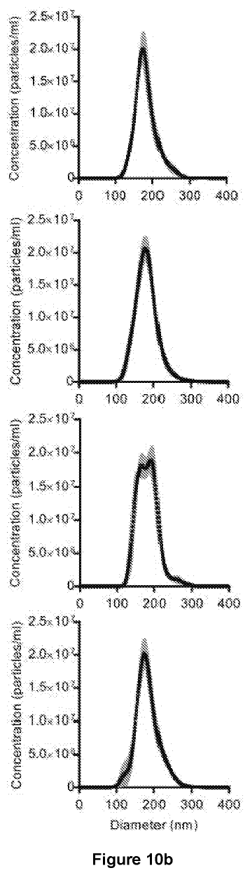

The inventors have devised a new strategy to purify large-scale amounts of EVs from RBCs at low cost. RBCs were obtained from group O blood of healthy donors and treated with calcium ionophore overnight. The purification of RBCEVs was optimized with sequential centrifugation steps including the removal of protein contamination using a 60% sucrose cushion that yielded a homogenous population of EVs with an average diameter of ˜140 nm and a poly-dispersity index ˜0.07, determined using a Nanosight particle analyzer and Zetasizer (FIG. 1a and FIG. 9a, b). Each unit of RBCs, isolated from ˜200 ml blood, yielded 5-10×1013 EVs. These EVs were negatively charged with a Zeta potential of −11.5 mV on average (FIG. 9c). The morphology of the EVs appeared heterogeneous under a transmission electron microscope (TEM), with a mixture of both small exosome-like and large microvesicle-like particles (FIG. 1b). Purified RBCEVs were enriched in EV markers (ALIX and TSG101) and hemoglobin A, the major RBC protein (FIG. 1c). In addition, RBCEVs were also enriched in Stomatin (STOM), a marker of RBCEVs, but completely lacked Calnexin (CANX), an endoplasmic reticulum marker which is common in many cell types but absent in RBCs (FIG. 1d). These data illustrated the identity and purity of RBCEVs; there was no contamination from other types of blood EVs. Moreover, the RBCEVs were stable after multiple freeze-thaw cycles. There was no aggregation or any significant change in the morphology, concentration, or size distribution of the EVs after 1-3 freeze-thaw cycles as determined by using both TEM and Nanosight particle analysis (FIG. 10a, b).

### Delivery of ASOs to Leukemia Cells Using RBCEVs

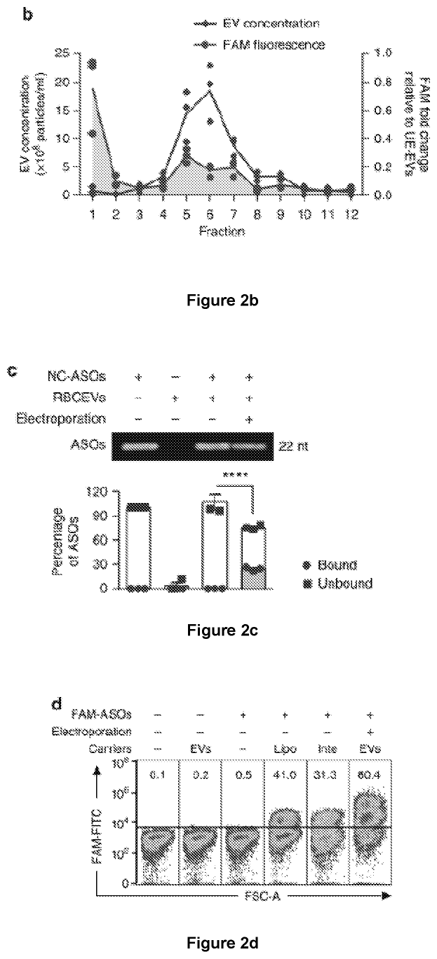

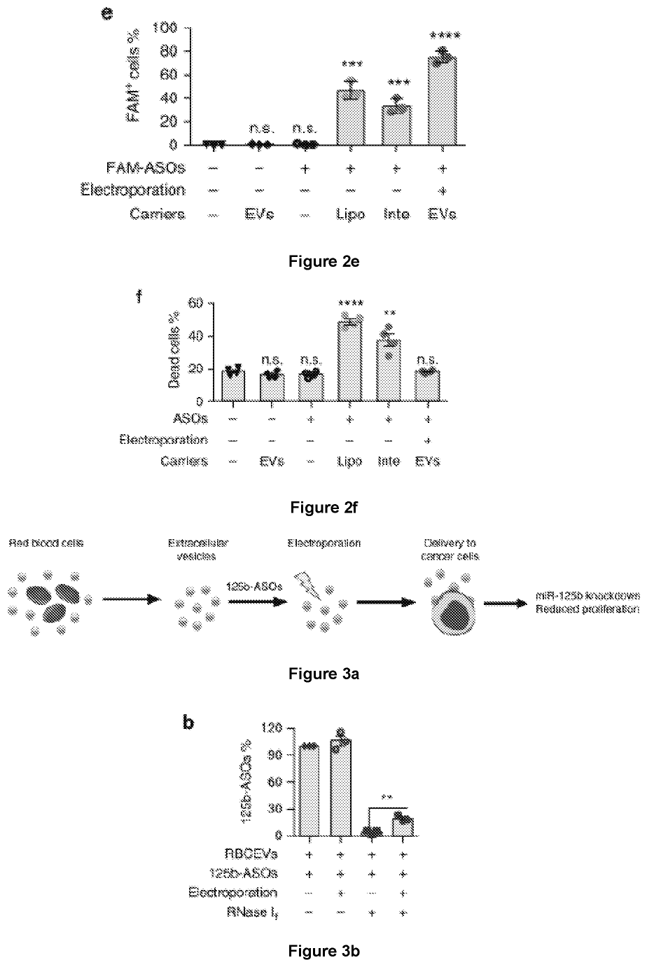

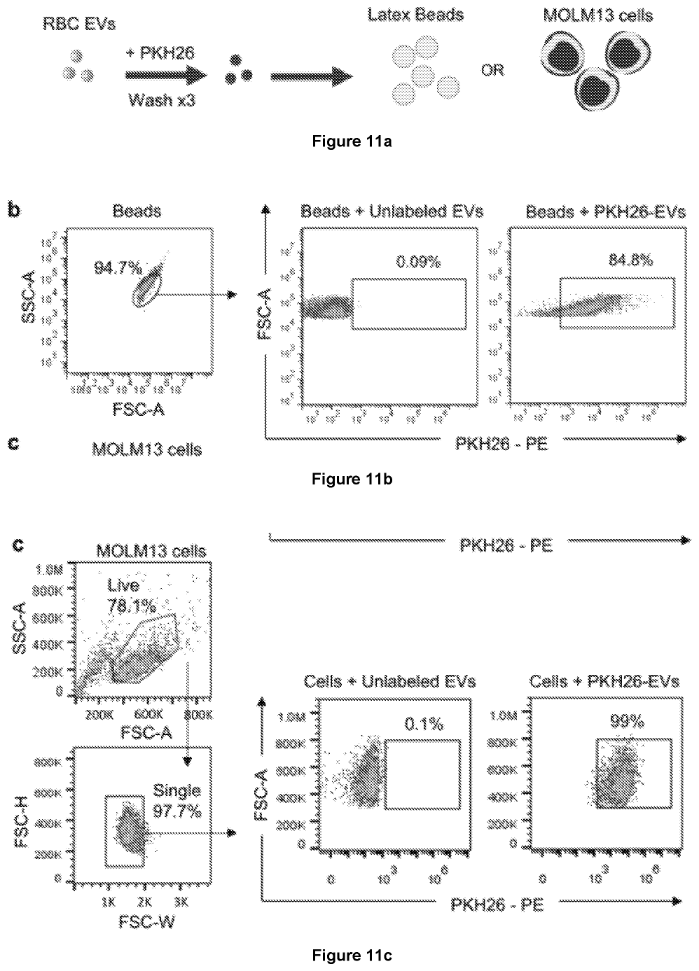

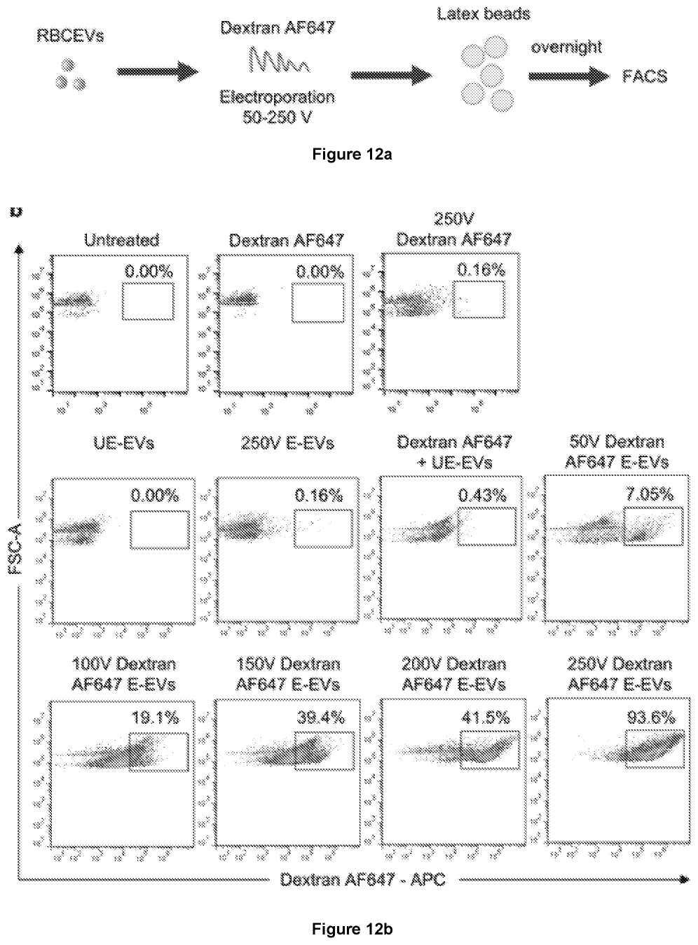

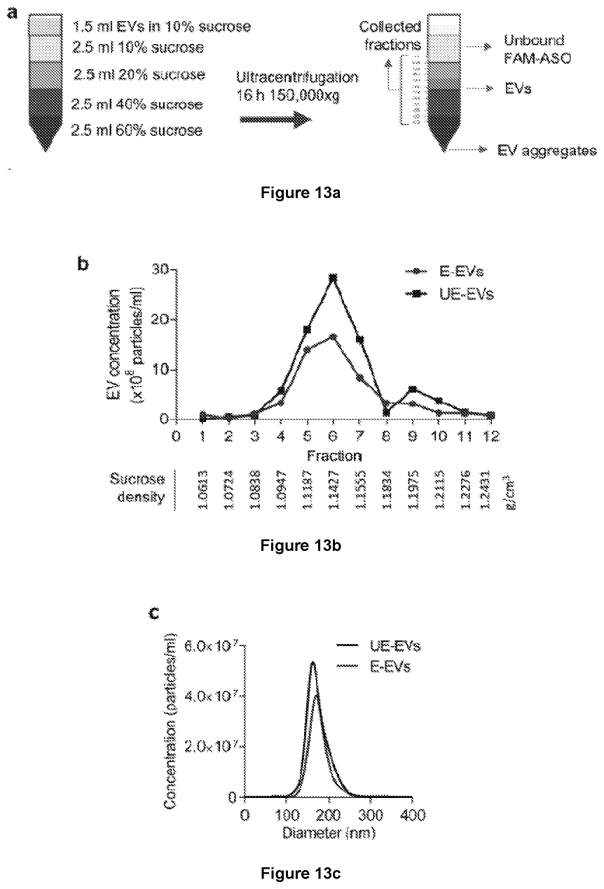

RBCEVs were taken up by leukemia cells at high efficiencies with no observable toxicity. After 24 h of incubation with RBCEVs, Western blot analysis of leukemia MOLM13 cells showed a clear uptake of Hemoglobin A, which was absent in the untreated cells (FIG. 1e). MOLM13 cells became ˜99% fluorescent positive after a 24 h incubation with fluorescence-labeled EVs that was observed by both immunostaining and FACS (FIG. 1f, g and FIG. 11). This uptake was reduced by 60-70% when heparin was added together with RBCEVs to the cells, suggesting that RBCEV uptake was dependent on heparan sulfate proteoglycans (FIG. 1g, h). The inventors optimized the electroporation of RBCEVs with Alexa Fluor® 647 labeled Dextran and obtained up to 93.6% fluorescent EVs at the voltage of 250 V (FIG. 12). Subsequently the inventors electroporated RBCEVs with a FAM-labeled scrambled negative control ASOs (FAM-NC-ASOs) with 2′ Omethyl modification at every nucleotide (FIG. 2a). To compare the density of unelectroporated and electroporated RBCEVs, the inventors layered 3.3×1012 RBCEVs on top of a 10-60% sucrose gradient and separated the EVs using ultracentrifugation at 150,000×g for 16 h (FIG. 13a). Both unelectroporated and electroporated RBCEVs were concentrated in fraction 5-7, at the interphase of the 20 and 40% sucrose layers, with the density of ˜1.12-1.16 g/cm3 consistent with a previous report (FIG. 13b). Unelectroporated and electroporated RBCEVs in fraction 6 exhibited the same size distribution (FIG. 13c). However, the concentration of electroporated RBCEVs was lower than unelectroporated RBCEVs in fraction 5-7 (FIG. 13b). A red pellet was found at the bottom of the gradients of the electroporated (but not the unelectroporated) RBCEVs suggesting that some of the electroporated EVs formed aggregates. FAM fluorescence was enriched in fraction 5 to 7 of RBCEVs electroporated with FAM-labeled ASOs, indicating that RBCEVs were loaded successfully with FAM ASOs (FIG. 2b). Unbound FAM ASOs in fraction 1 emitted a higher FAM intensity than the EV-enriched fractions, suggesting that only a portion of FAM ASOs were loaded into the RBCEVs (FIG. 2b).

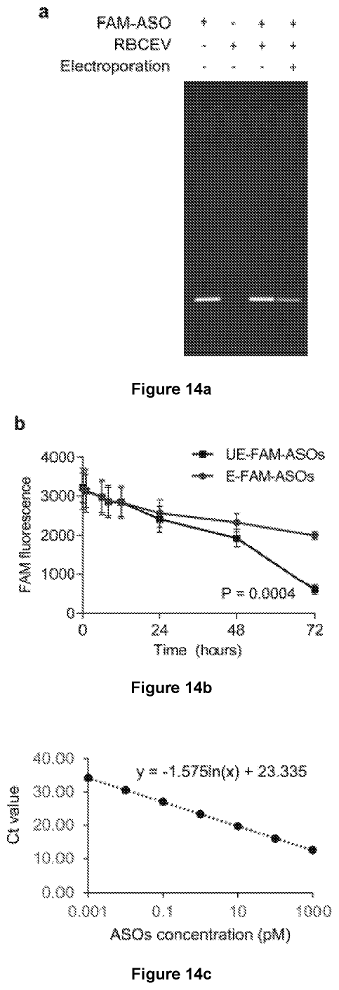

To improve estimate of RNA loading into electroporated RBCEVs, the inventors separated unbound ASOs from the electroporated RBCEVs using a 10% native gel and found that ˜76% of the ASOs migrated into the gel from the 8.25×1011 ASO electroporated RBCEV sample relative to the total 200 pmol ASOs (FIG. 2c). Hence, ˜24% of the ASOs were loaded into the RBCEVs by electroporation. Similar loading efficiencies were observed based on the FAM fluorescence in RBCEVs electroporated with FAM ASOs (FIG. 14a). It is noteworthy that unbound ASOs appeared as a single band in all samples including the electroporated RBCEVs and no FAM signal was detected in the wells. Hence, the electroporation did not cause any aggregation of the ASOs, unlike the aggregation of Cy3 or Cy5-labeled oligonucleotides that were observed before. To test the RNA stability within RBCEVs in blood serum-like conditions, the inventors incubated FAM-ASO electroporated RBCEVs or an unelectroporated mixture of FAM ASOs and RBCEVs with 50% FBS, which contained various nucleases at 37° C. for 72 h. The fluorescent signal of electroporated FAM ASOs declined at a significantly lower rate than unelectroporated FAM ASOs (FIG. 14b). This data suggested that the FAM ASOs were protected from extracellular nuclease-mediated degradation after incorporation into RBCEVs.

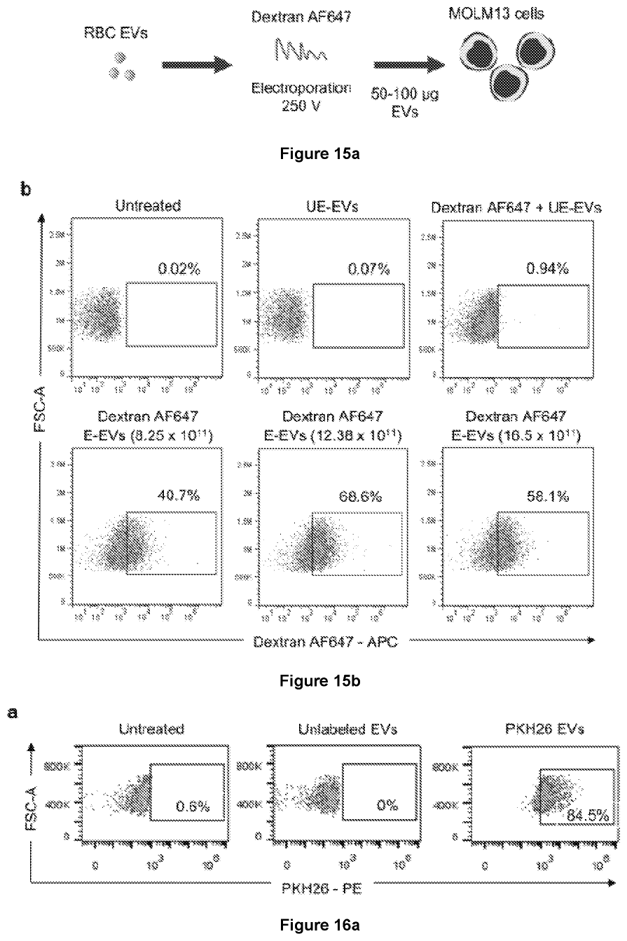

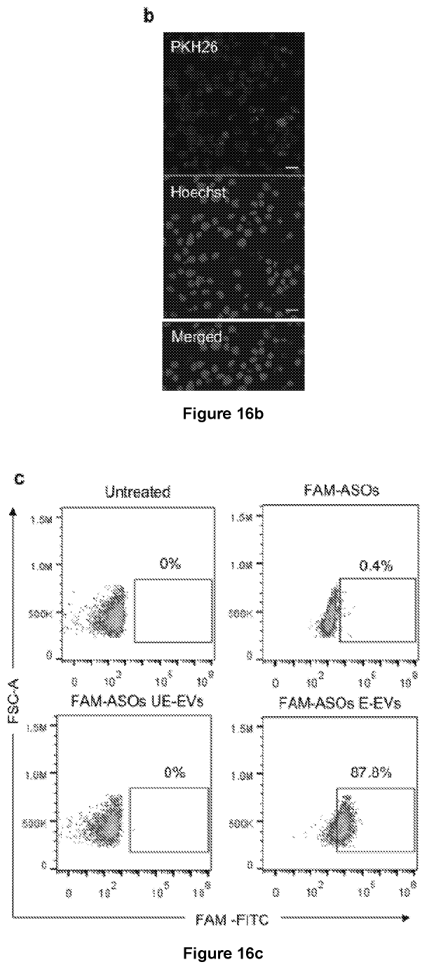

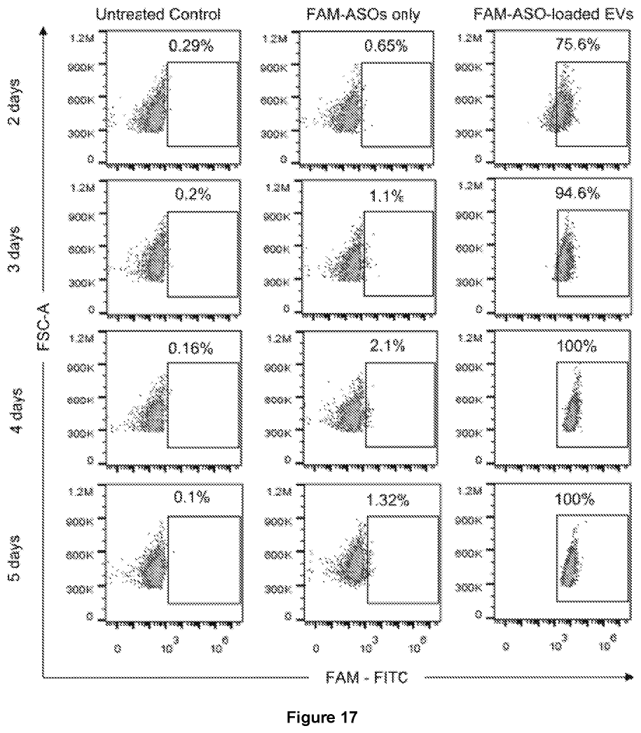

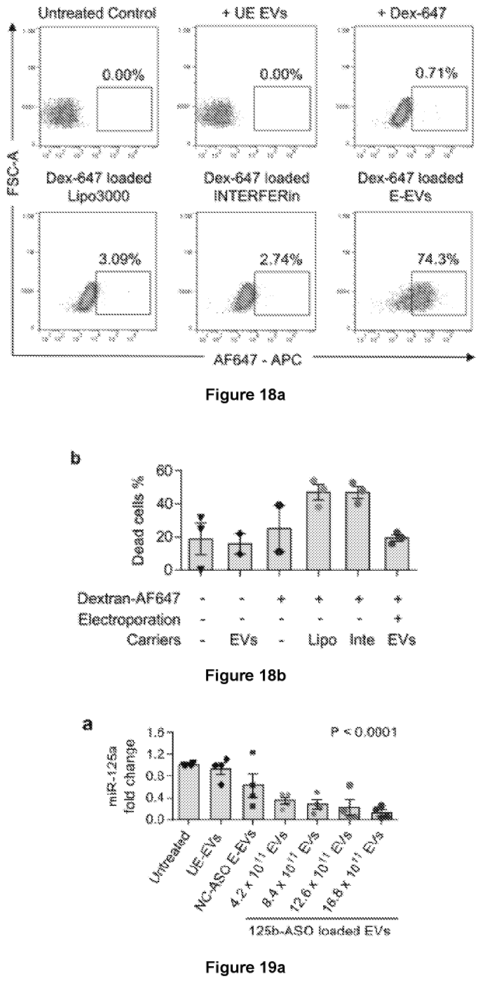

The inventors consistently observed 70-80% uptake of FAM-labeled ASOs and Alexa Fluor® 647 labeled Dextran in leukemia MOLM13 cells after 24-hourtreatments with ˜12×1011 RBCEVs for 5×104 cells, which was the optimal dose (FIG. 2d and FIG. 15). The uptake of FAM ASOs was ˜88% corresponding to ˜85% uptake of fluorescent-labeled RBCEVs in another leukemia cell line, NOMO1 (FIG. 16a-c). FAM fluorescence was observed in ˜0.4% MOLM13 and NOMO1 cells after 24 h of incubation with unencapsulated (unbound) FAM ASOs (FIG. 2d, e and FIG. 16c). Over 4 days, this signal increased to 2.1% of MOML13 cells suggesting that the unencapsulated FAM ASOs were taken up slowly by a tiny population of MOLM13 cells via gymnotic delivery (FIG. 17). After 4 days, the percentage of FAM-positive MOLM13 cells did not increase further with FAM-ASO treatment. However, during the same period of time, the delivery of FAM ASOs by RBCEVs occurred at a much higher rate in MOLM13 cells, from 75% after 2 days to 100% FAM-positive cells after 4 days of incubation (FIG. 17). These data indicated that RBCEVs conferred remarkable delivery efficiencies, since leukemia cells and most blood cells are considered cell types that are very difficult to transfect. Commercial transfection reagents, including Lipofectamine™ and INTERFERin®, could only produce ˜3% uptake of Dextran and 33-46% uptake of ASOs in MOLM13 cells after 24 h of transfection (FIG. 2d, e and FIG. 18a). Moreover, RBCEVs did not cause any toxicity to the cells. This is in contrast to the ˜20-30% increase in cell death caused by Lipofectamine™ 3000 and INTERFERin® (FIG. 2f and FIG. 18b). Therefore, RNA delivery by RBCEVs show higher efficiency and lower toxicity in leukemia cells, compared to current transfection vehicles.

Inhibition of miR-125b Using 125b-ASO-Loaded RBCEVs

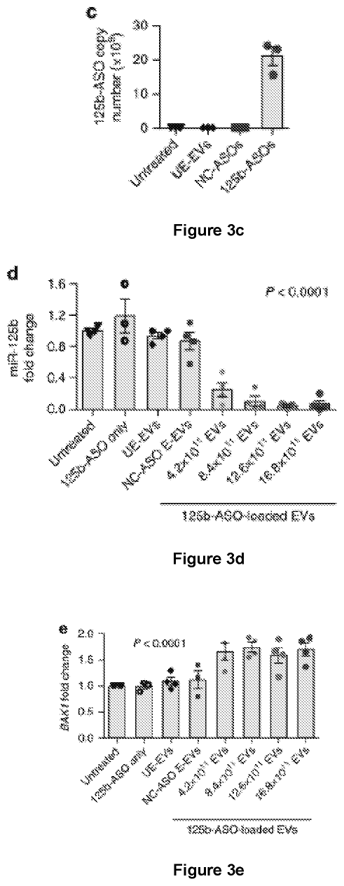

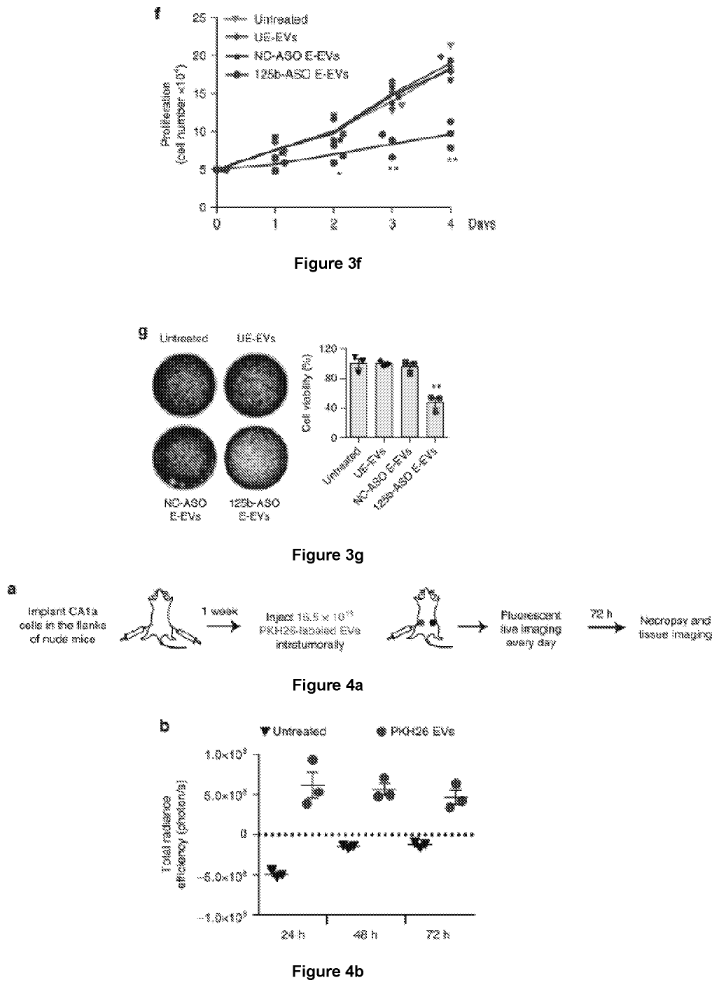

The inventors further investigated the therapeutic potential of RBCEVs in delivering ASOs that antagonizes miR-125b, a well-known oncogenic microRNA in leukemia cells, prostate cancer, and breast cancer. In particular, miR-125b is an oncomiR in refractory cancers such as acute myeloid leukemia and chemoresistant breast tumors, both of which are difficult to treat. The inventors have shown that miR-125b promotes the survival of cancer cells by repressing multiple genes in the p53 tumor suppressor network. These studies suggested that miR-125b is a potential drug target for cancer treatment. However, no effective therapy has been developed to target miR-125b yet. Here, the inventors electroporated anti-miR-125b ASOs (125b-ASOs) into RBCEVs then quantified the loading of 125b-ASOs in the EVs and the delivery of the ASOs to MOLM13 cells (FIG. 3a). Treatment of 6.2×1011 electroporated RBCEVs with RNase If led to a degradation of ˜80% 125b-ASOs, relative to the untreated ASOs, quantified using a sequence-specific Taqman qRTPCR; whereas, the same amount of ASOs in an unelectroporated mixture with RBCEVs was completely degraded (FIG. 3b). This data suggests that approximately 20% of the ASOs (-24×1012 copies) were loaded into RBCEVs by electroporation and thus, protected from the

RNase. To quantify the copy number of 125b-ASOs, the inventors generated a standard curve of the ASOs amplification using Taqman qRT-PCR (FIG. 14c). Based on this curve, the inventors found ˜21×109 copies of 125b-ASOs in MOLM13 cells after a 72-h-incubation with 12×1011 125b-ASO-loaded RBCEVs (FIG. 3c).

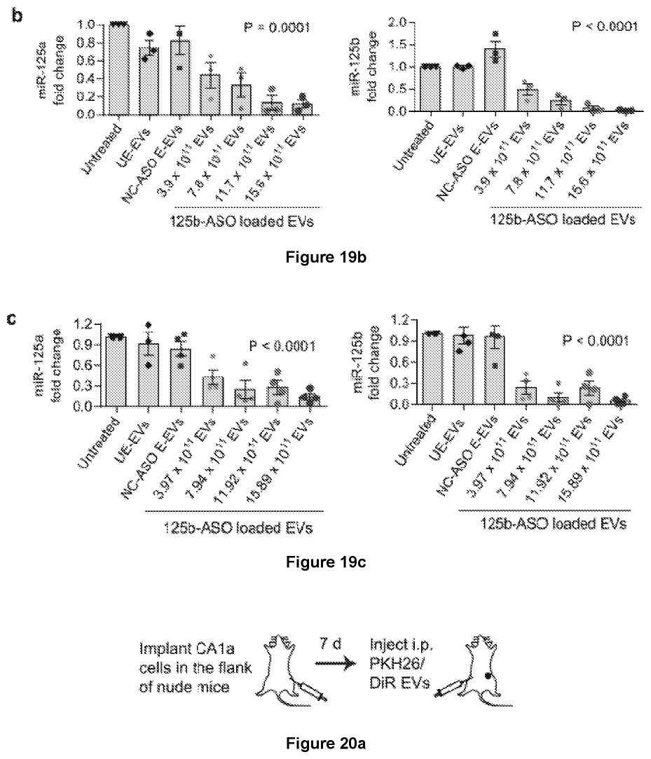

With the uptake of 125b-ASOs, the level of miR-125b was suppressed by 80-95% in MOLM13 cells in a dose-dependent manner relative to U6b RNA quantified using Taqman qRT-PCR (FIG. 3d). This result was confirmed using miRCURY-LNA qRTPCR with miR-103a as the internal control (FIG. 23a). miR-125a, the homolog of miR-125b, was also suppressed by 50-80% in a dose-dependent manner (FIG. 19a). The same effects were observed in NOMO×1-cells (FIG. 19b). The inhibition of the miR-125 family led to a significant increase in BAK1, a target of the miR-125 family that the inventors previously identified (FIG. 3e). The ASOs alone did not have any effect on miR-125b or BAK1 expression (FIG. 3d, e). Treatment with 125b-ASO-loaded RBCEVs also significantly dampened the growth of MOLM13 cells after 3-4 days of incubation (FIG. 3f). To test the function of miR-125b in another cancer type, the inventors applied the same treatment to human breast cancer MCF10CA1a (CA1a) cells. The inventors found a significant knockdown of miR-125a/b and reduced survival of CA1a cells after treatment with 125b-ASO-loaded RBCEVs (FIG. 19c and FIG. 3g).

In vivo Distribution of RBCEVs in a Breast Cancer Model

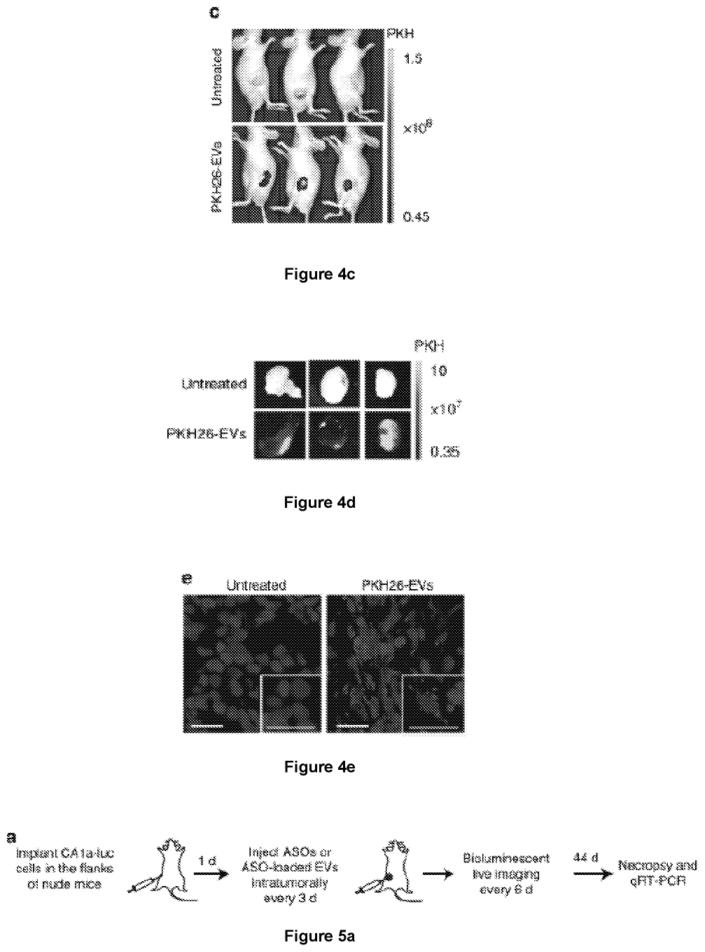

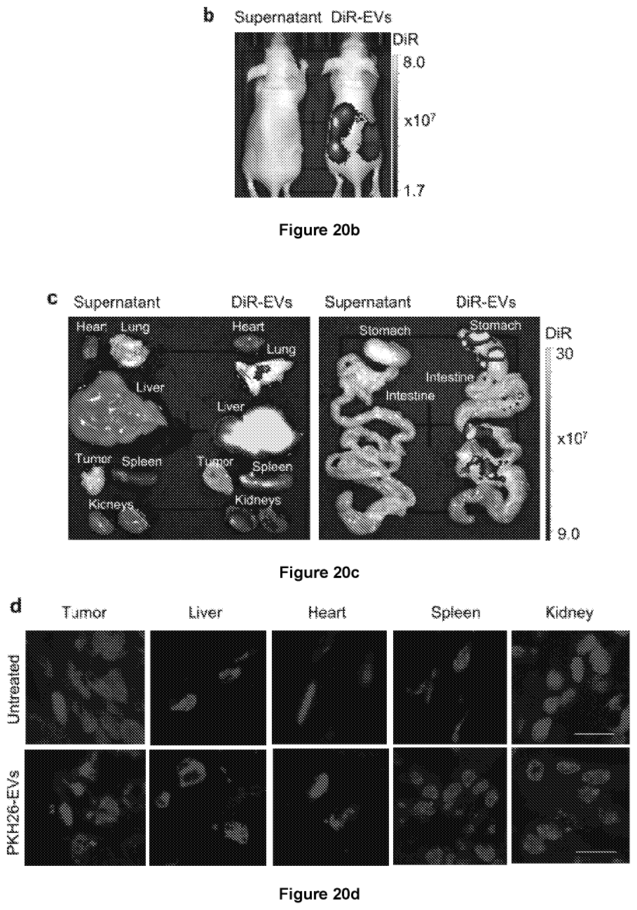

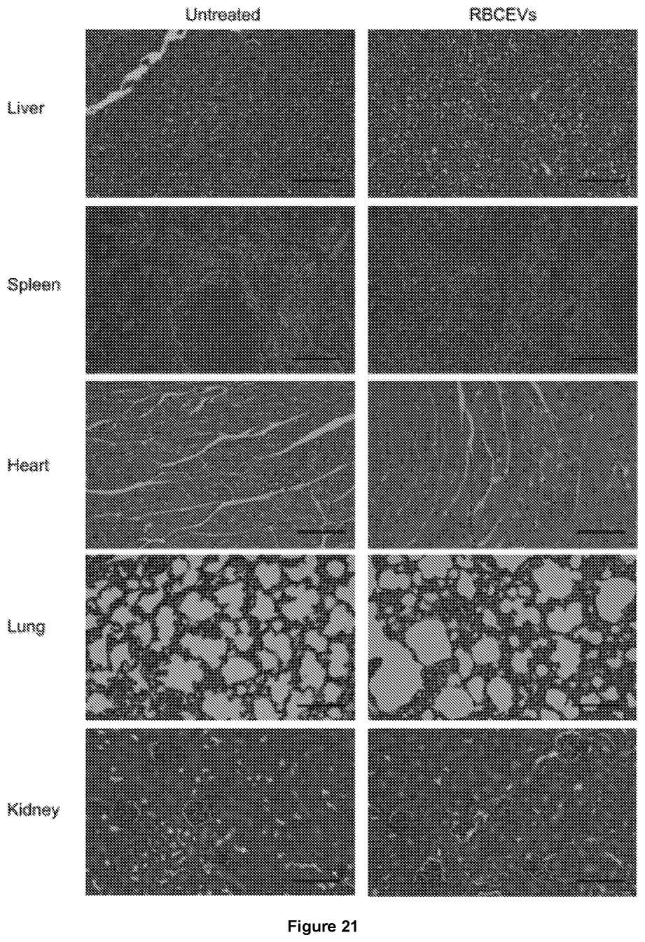

We tested the uptake and distribution of RBCEVs in vivo using the xenograft model of breast cancer CA1 cells, known to be very aggressive and metastatic. Luciferaseexpressing CA1 a cells were implanted subcutaneously in the left and right flanks of female nude mice (FIG. 4a). After 1 week (as the tumors approached 7 mm in diameter), the inventors injected the left tumors with PKH26-labeled RBCEVs. The fluorescence signal became concentrated in the tumors and gradually declined over time (FIG. 4b), observed using an in vivo imaging system (IVIS). After 72 h, the PKH26 signal was still detectable in the tumors but undetectable in other parts of the body (FIG. 4c, d). High resolution images of the tumor sections confirmed the internalization of PKH26-labeled RBCEVs by cells in the tumors (FIG. 4e). By contrast, when the inventors injected PKH26 or DiR-labeled EVs intra-peritoneally (i.p.) into nude mice bearing flank tumors, PKH26, or DiR fluorescence was widely dispersed in the body (FIG. 20a, b). DiR signal was enriched in the liver, spleen, stomach, intestine, kidneys, and lung (FIG. 20b, c). In cryosections of tissues from PKH26-EV i.p. injected mice, the inventors found some RBCEV uptake in the tumor after i.p. injection, but much less than intratumoral injection (FIG. 20d). The inventors did not observe any inflammation, nor changes in morphology and cellular contents of the liver and other organs after these injections (FIG. 21). Hence, local injection delivered RBCEVs more effectively to the tumors while systemic administration distributed RBCEVs to multiple organs without any significant cytotoxicity in nude mice.

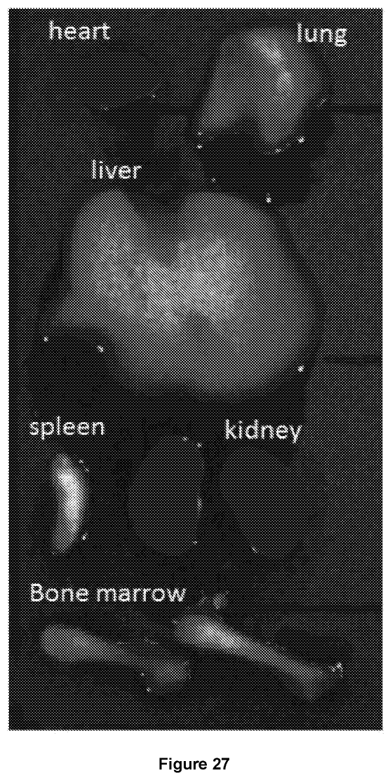

The biodistribution of RBCEV in vivo was also tested in NOD scid gamma (NSG) mouse (FIG. 27) injected i.v. with RBCEVs (FIG. 27). Based on the biodistribution of RBCEVs, i.e. biodistribution by i.p. injection (FIG. 20) and biodistribution by i.v. injection (FIG. 27), they can treat diseases in the liver, bone marrow, lung, spleen, stomach and intestine.

Intratumoral Injection of 125b-ASO-Loaded RBCEVs Suppresses Breast Cancer

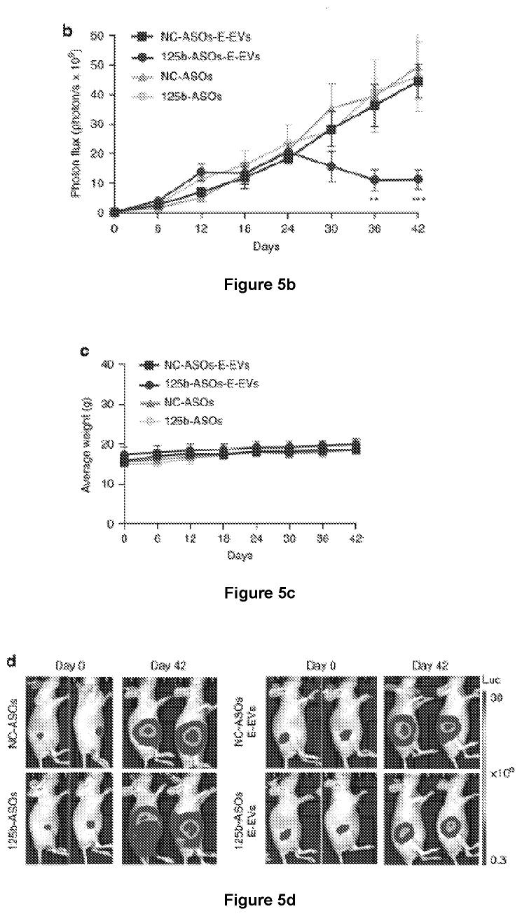

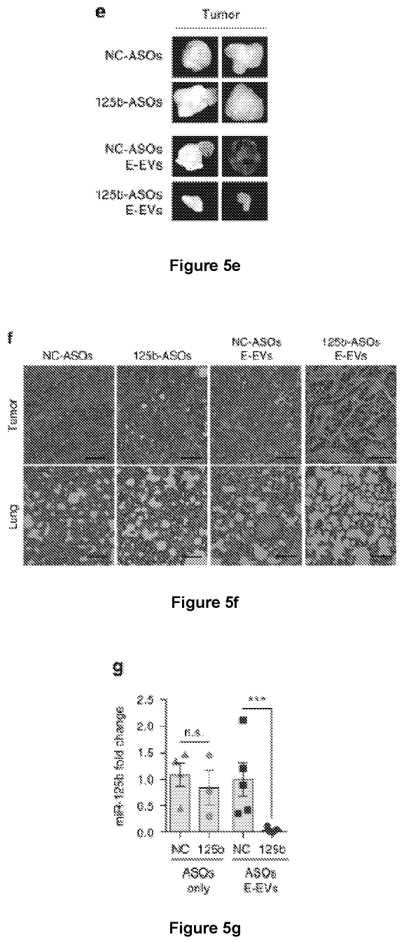

After validating the RBCEV platform's potential utility in vivo, the inventors used it to target miR-125b, which has not been tested for its role in breast tumorigenesis in vivo. The inventors delivered 125b-ASO-loaded RBCEVs into luciferase-labeled CA1 a tumors by intratumoral injections every 3 days (FIG. 5a). Breast tumor growth was significantly dampened by 125b-ASO-loaded RBCEVs, as observed from the decrease in tumor bioluminescence compared to the NC-ASO-loaded RBCEVs after 30-42 days of treatment (FIG. 5b, d). Injection of 125b-ASOs without RBCEVs did not result in any significant change in tumor growth compared to the NC-ASOs treatment. There was no significant difference in overall weight between the controls and 125b-ASO-loaded-RBCEV treated mice, suggesting that 125b-ASO-loaded RBCEVs induced tumor shrinkage specifically, without causing overall weight loss and toxicity (FIG. 5c). When harvested, the 125b-ASO-loaded-RBCEV treated tumors were smaller than the controls (FIG. 5e). Hematoxylin and eosin (H & E) staining of tumor sections showed that 125b-ASO-loaded-RBCEV treated tumors were also less invasive, and less metastasis was observed in the lung (FIG. 5f). Remarkably, miR-125b was reduced by ˜95% in the 125b-ASO-loaded-RBCEV treated tumors (FIG. 5g). These data suggested that RBCEVs were avidly taken up by breast cancer cells in vivo, and that RBCEVs can deliver therapeutic ASOs to effectively antagonize oncomiRs and suppress tumorigenesis without any observable side effects.

Systemic Injection of 125b-ASO-Loaded RBCEVs Suppresses AML Progression

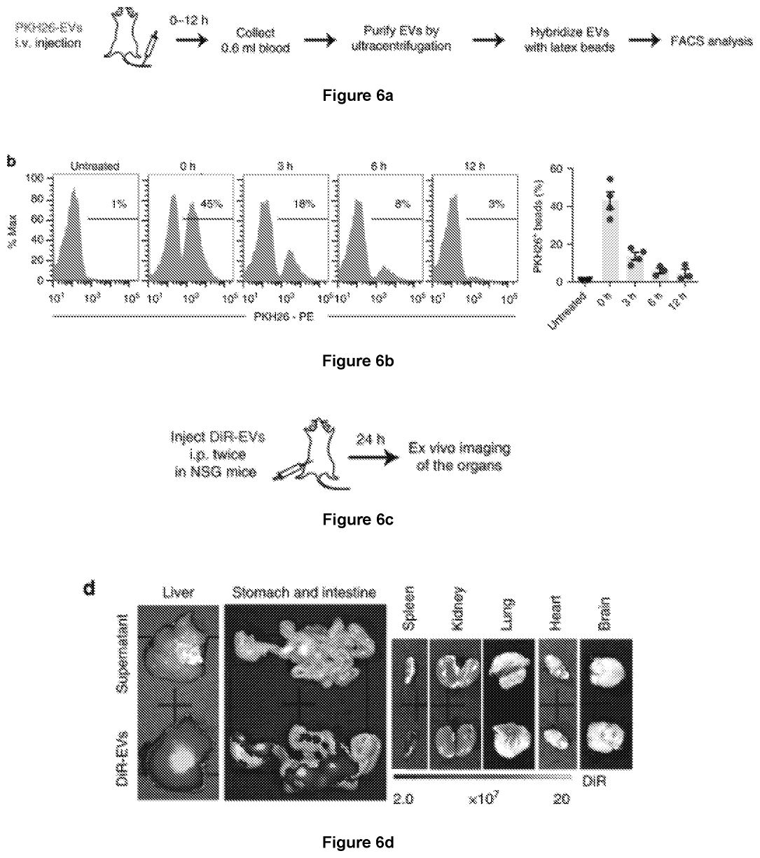

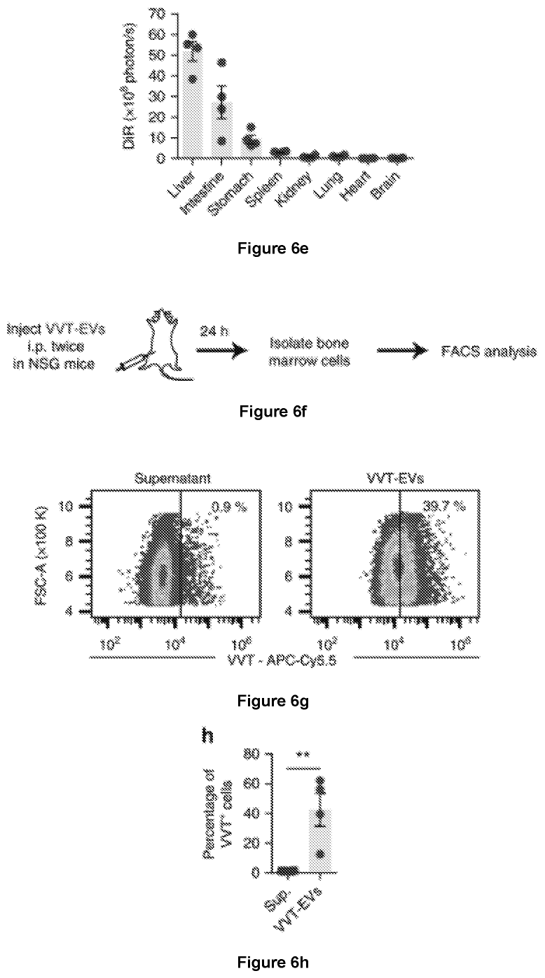

The inventors have shown that RBCEVs are robust vehicles that delivered 125b-ASOs readily to AML cells for effective inhibition of miR-125b function in vitro. To test the functional efficacy of 125b-ASO-loaded RBCEVs in vivo, the inventors sought to establish a xenograft model of AML in NSG mice. First, the inventors determined the distribution of RBCEVs in the circulation of these mice following systemic administration of the EVs (FIG. 6a). Immediately after an intravenous (i.v.) injection of 3.3×1012 PKH26-labeled RBCEVs (hereafter, referred as one dose), the inventors found more than 40% circulating EVs positive for PKH26 (FIG. 6b). The percentage of PKH26-positive EVs declined over 6 h and remained at 3 4.5% after 12 h. The decrease in circulating human RBCEVs suggested that some of the EVs were taken up by the mouse tissues over time. To confirm that RBCEVs can be distributed to various organs of NSG mice by i.p. injection, the inventors administered two doses of DiR-labeled RBCEVs i.p. 24 h apart. 24 h after the second dose, the inventors found bright DiR signals in the liver, spleen, stomach, and intestine using the fluorescence IVIS (FIG. 6c-e). Robust delivery of RBCEVs to the internal organs suggested that i.p. administered RBCEVs could effectively treat leukemia cells in the liver and spleen where leukemia usually develop. Due to the blocking of DiR signals by dense bone and the unavailability of microscope filters or cytometer filters with long excitation/emission wavelengths (750/780 nm for DiR), the inventors were unable to detect DiR from the excised bone or bone marrow aspirates. Because bone marrow is the primary compartment that leukemia cells home into, the inventors attempted to determine the uptake of RBCEVs by bone marrow cells using RBCEVs labeled with Vivo-Track-680 (VVT), a near-infrared membrane dye that is detectable by FACS (FIG. 6f). Indeed, VVT fluorescence was detected in ˜40% of the bone marrow cells from the NSG mice injected i.p. with VVT labeled RBCEVs using FACS analysis (FIG. 6g, h). Hence, RBCEVs were robustly taken up by bone marrow cells and could deliver therapeutic molecules for leukemia treatment in vivo.

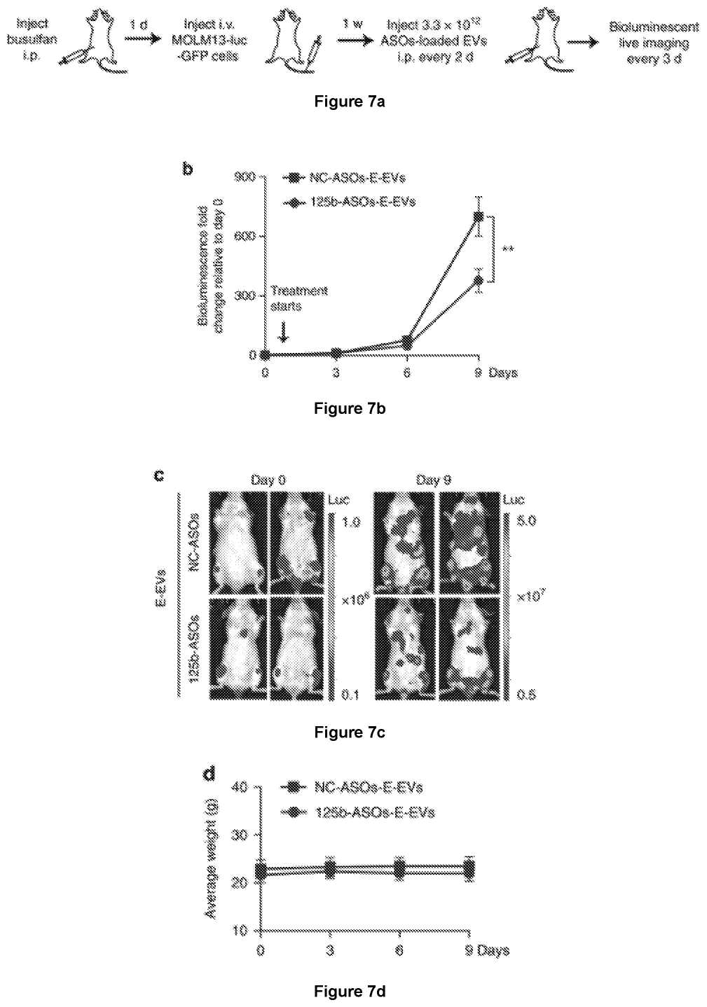

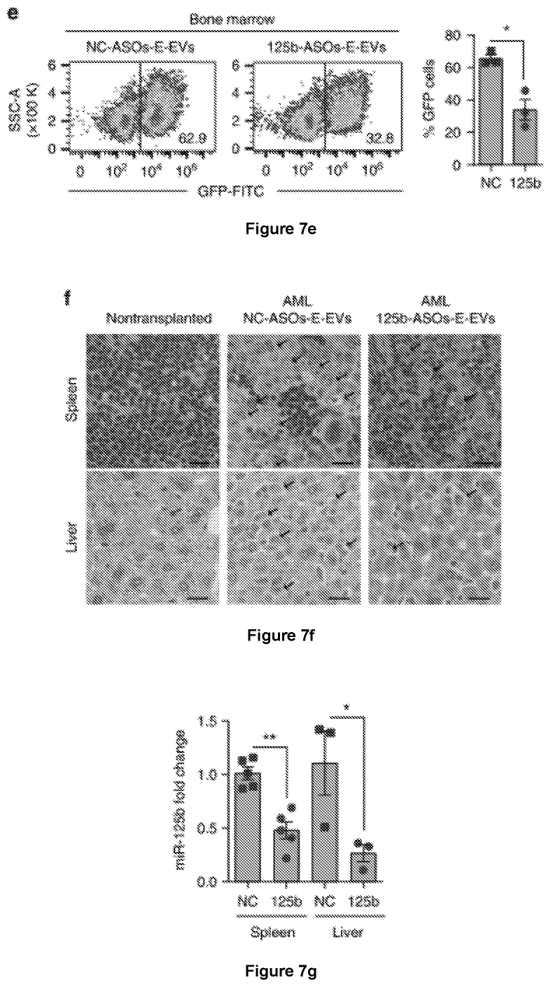

Subsequently, the inventors generated AML xenografts by injecting luciferase-GFP labeled MOLM13 cells in the tail vein of busulfan-conditioned NSG mice (FIG. 7a). After 1 week when leukemic bioluminescent signals became visible, the inventors treated the mice with one dose of 125b-ASO-loaded RBCEVs every other day. The leukemia developed very rapidly so the inventors could treat the mice for only 9 days before the control group paralyzed and died (usually 18-20 days after the cell inoculation). On day 9, the leukemic bioluminescence in mice treated with 125b-ASO-loaded RBCEVs decreased significantly compared to the control group (FIG. 7b). The control mice became very weak while the 125b-ASO-EVs treated mice were still active. The leukemic bioluminescent signals spread all over the mice′ bodies, accumulating highly in the bone marrow, liver, and spleen of control mice, compared to the 125b-ASO-loaded-RBCEVs treated group (FIG. 7c). The inventors could not assess the effect of the treatments on the overall survival of the mice due to restrictions defined by our institutional ethics committee. All the mice were killed on day 9 except for 2 control mice that died on day 8. GFP+leukemia cells accounted for 63-70% cells in the bone marrow of the controls but reduced to 27-46% in the treated mice albeit there was no change in the body weight (FIG. 7d, e). H & E staining revealed extensive infiltration of leukemia cells in the liver and spleen of the control group while less leukemia cells were found in the 125b-ASO-loaded-RBCEV treated group (FIG. 7f). Moreover, qPCR analysis showed a significant knockdown of miR-125b in the spleen and liver (FIG. 7g). These data indicated that 125b-ASOs delivered by RBCEVs was taken up by leukemia cells and effectively suppressed leukemia progression in this model. Thus RNA inhibition using systemic administration of ASO-loaded RBCEVs may represent a new approach for leukemia treatment.

Genome Editing Mediated by Cas9 mRNA and gRNA-Loaded RBCEVs

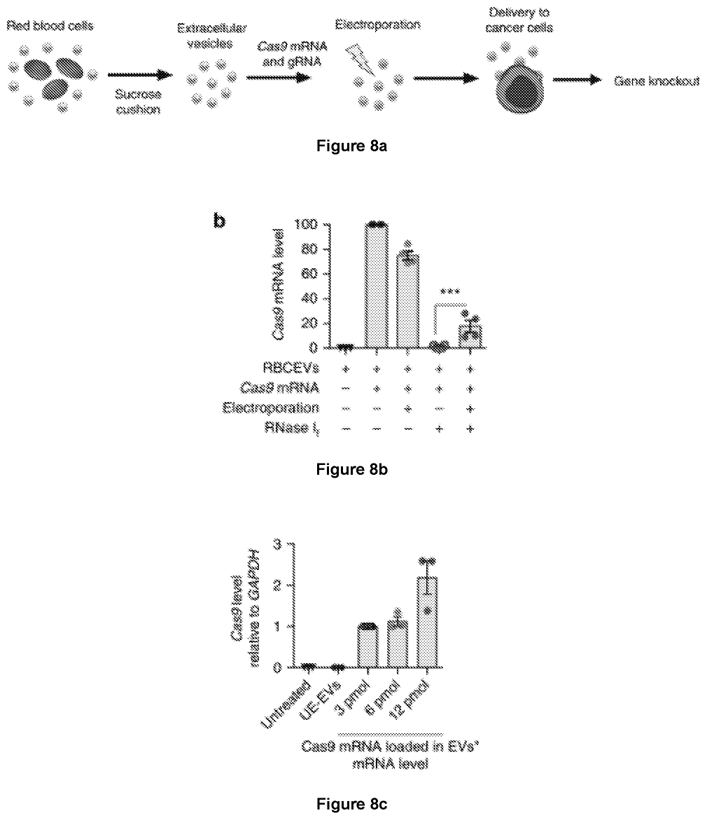

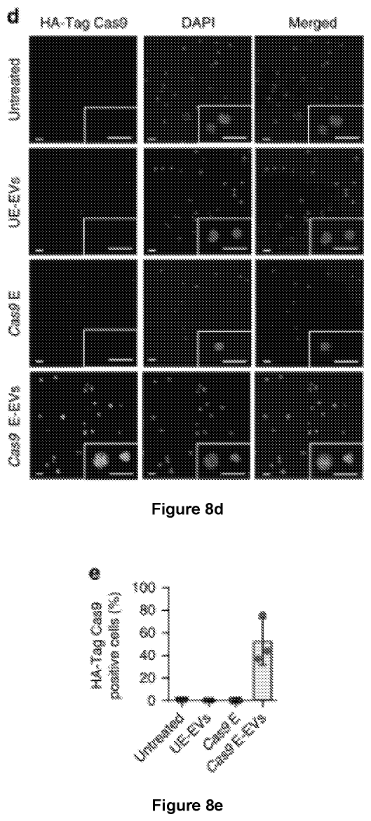

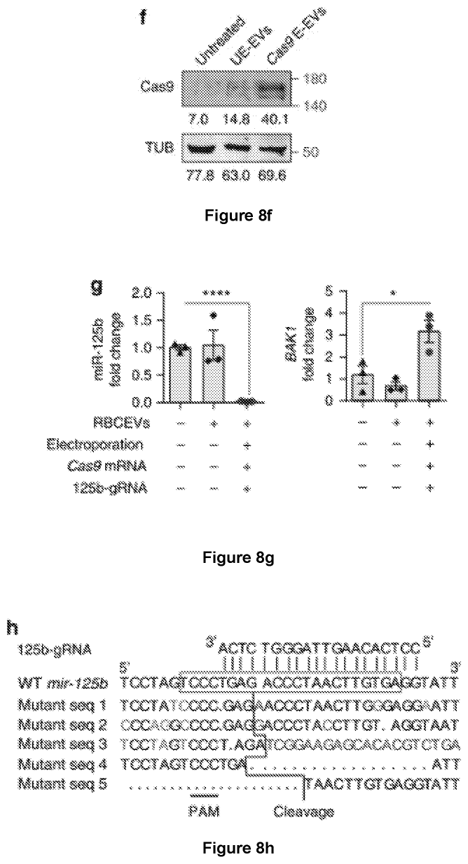

Furthermore, the inventors validated the RBCEV platform for gRNA-mediated genome editing with CRISPR-Cas9 (FIG. 8a). To test the feasibility of mRNA delivery using RBCEVs, the inventors electroporated HA-tagged Cas9 mRNA (4,521 nucleotides) into RBCEVs, and used them to treat MOLM13 cells. The inventors first quantified the loading of Cas9 mRNA in RBCEVs using qPCR. Electroporated Cas9 mRNA was protected by RBCEVs from RNase If mediated degradation while unelectroporated Cas9 mRNA was completely degraded (FIG. 8b). Specifically, about 18% of Cas9 mRNA was loaded and protected in RBCEVs. The inventors detected abundant Cas9 mRNA in MOLM13 cells after a 24-h incubation of the cells with Cas9 mRNA electroporated RBCEVs, whereas the cells treated with unelectroporated RBCEVs had no detectable Cas9 mRNA (FIG. 8c and FIG. 23b). Cas9 protein was efficiently expressed in the nuclei of ˜50% MOLM13 cells at 48 h post-treatment, detected using immunostaining with an anti-HA tag antibody (FIG. 8d, e). Western blot analysis confirmed the expression of Cas9 protein using an anti-Cas9 antibody (FIG. 8f).

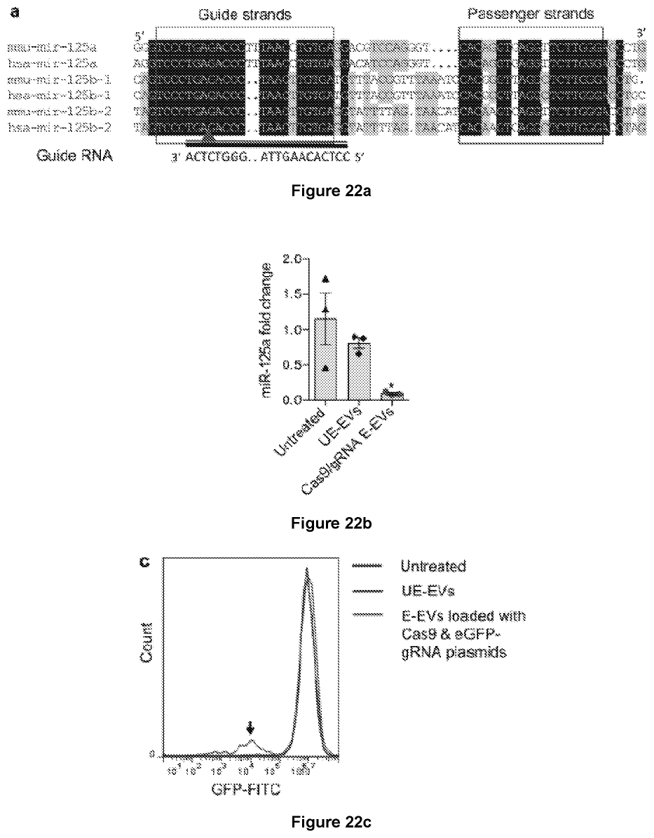

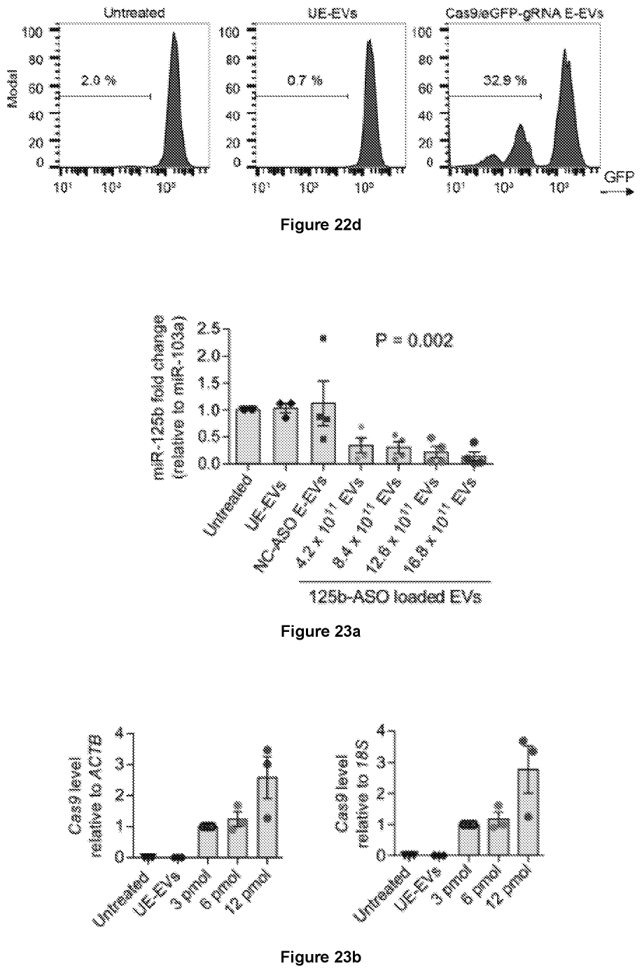

Subsequently, the inventors designed a gRNA targeting the human mir-125b-2 locus with potential mutation site in the seed sequence of the miRNA (FIG. 22a). This gRNA may bind to the other loci of the miR-125 family due to sequence similarity. Treatment of MOLM13 cells with RBCEVs loaded with Cas9 mRNA and 125b-gRNA resulted in ˜98% reduction of miR-125b expression and 90% reduction of miR-125a after a 2-day treatment (FIG. 8g and FIG. 22b). As a consequence, BAK1 was upregulated by approximately three fold (FIG. 8g). Sequencing data confirmed a cleavage site 3-8 nucleotides apart from the protospacer adjacent motif (PAM) sequence in each of the mutant clones (FIG. 8h). Insertions and deletions of different sizes that disrupted the mature miR-125 sequence were found at the cleavage sites (FIG. 8h). The rapid and high efficiency of miR-125a/b suppression was probably due to the short half-life of miR-125a/b, in addition to genome editing. These data suggest that RBCEVs are able to deliver a functional CRISPR-Cas9 genome editing system into leukemia cells effectively.

To test if RBCEVs can also deliver DNA plasmids, the inventors electroporated two plasmids expressing Cas9 and GFP gRNA into RBCEVs and treated 293T-eGFP cells for a week. EGFP knockout was observed in only ˜10% cells (FIG. 22c), likely due to the large sizes of the DNA plasmids. RBCEVs were also electroporated with a combination of Cas9 mRNA and anti-eGFP gRNA at a 6:50 molar ratio. The Cas9 mRNA/gRNA-loaded RBCEVs led to a complete loss of eGFP in ˜32% NOMO1-eGFP cells (FIG. 22d). Hence, RBCEVs can be used to deliver RNAs and DNAs for genome editing, albeit with lower efficiency for large DNA plasmids and higher efficiency for RNAs.

### RBCEVs in the Treatment of Lymphoma

The uptake of Bodipy-labeled RBCEVs by lymphoma B95-8 cells was studied by the inventors (Supplementary FIG. 20) and showed significant uptake of Bodipy-labeled RBCEVs by B95-8 cells, thus showing that RBCEVs may be used in the treatment of lymphoma.

### Discussion

Taken together, the data demonstrates the use of RBCEVs as a versatile delivery system for therapeutic RNAs, including short RNAs such as ASOs and gRNAs, as well as long RNAs such as Cas9 mRNAs. ASOs and CRISPR-Cas9 can be designed and programmed to target any gene of interest, including undruggable targets such as oncomiRs and transcription factors, for therapeutic purposes. Previously, several research groups have illustrated the advantages of using EVs for RNA delivery, but their EVs were generated from fibroblasts and dendritic cells that are not as readily available from all subjects. EVs from whole plasma are more abundant and easier to obtain, but these EVs are derived from many cell types including nucleated cells, which still pose a risk for horizontal gene transfer.

The RBCEV platform has several features that are more suitable for clinical applications. First, blood units are readily available from existing blood banks and even from the patients' own blood for allogeneic and autologous transfusion, respectively. A large number of RBCs (˜1012 cells/L) are available in each blood unit. Hence, there is no need to expand the cells in culture and risk any accrual of mutations in vitro, and no cGMP-qualified media or supplements are required. Second, large-scale amounts (1013-1014) of EVs can be purified from RBCs, after the treatment with calcium ionophore, thus providing a scalable strategy to obtain EVs. Third, RBCEVs are safe, as the enucleated RBCs are homogeneously devoid of DNA, unlike EVs from nucleated cell types which pose potential risks for horizontal gene transfer and unlike plasma EVs that are heterogeneous with unpredictable contents. For allogeneic treatments of cancer, RBCEVs are safer than plasma EVs, since cancer cells and immune cells are known to release a large amount of cancer promoting EVs into the circulation of cancer patients.

Moreover, RBCEVs are nontoxic, unlike the synthetic transfection reagents. RNAs are stable in RBCEVs and fully functional in recipient cells as shown by our in vitro and in vivo data for liquid and solid cancers. RBCEVs are likely to be non immunogenic, via matching of the blood types, unlike lentiviruses, adenoviruses, adeno-associated viruses, nanoparticles, and most synthetic transfection reagents. And the inventors have also shown that RBCEVs deliver RNAs to cells at a higher efficiency than two commonly used transfection reagents. The inventors are able to deliver not only short RNAs but also long mRNAs in RBCEVs. RBCEVs were successfully used to target a specific oncomiR gene, not only via steric blocking, but also via CRISPR-Cas9 genome editing, which has not been shown hitherto. RBCEVs from group O− Rh negative blood are ideal for universal treatments. Finally, the fact that RBCEVs can be frozen and thawed many cycles without affecting their integrity and efficacy, suggests that they can be developed into stable pharmaceutical products in future. Further development of RBCEVs coated with cancer-targeting peptides or antibodies could potentially deliver therapeutic RNAs to cancer cells specifically, and reduce adverse side effects in normal tissues.

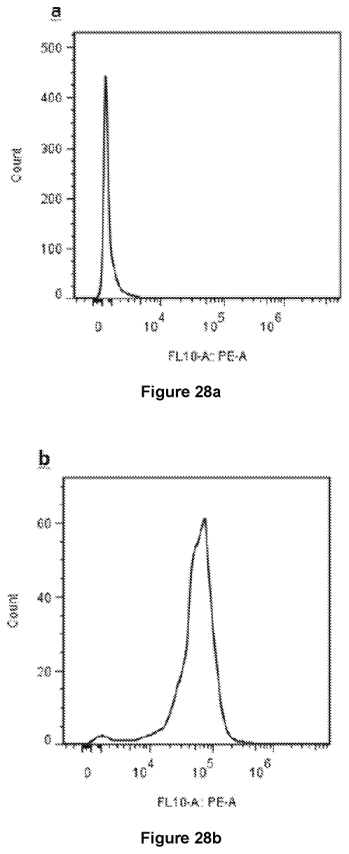

The experimental data obtained by the inventors show that RBCVs can be used to treat diseases in the liver, bone marrow, lung, spleen, stomach and intestine. Further, RBCEVs can be used in the treatment of cancers, including lung cancer, liver cancer, colorectal cancer, nasopharyngeal cancer, glioma, leukemia (FIG. 1-3, 7, 8), breast cancer (FIG. 3g, FIG. 3-5, sup FIG. 11b), kidney cancer (293T cells FIG. 14c), and lymphoma (FIG. 28)

### Methods

### Cell Culture

Acute myeloid leukemia MOLM13 and NOMO1 cells were originally from DSMZ Collection of Microorganisms and Cell Cultures (Braunschweig, Germany). Breast cancer MCF10CA1a (CA1a) cells were purchased from Karmanos Cancer Institute (Wayne State University, USA). HEK-293T cells were obtained from the American Type Culture Collection (ATCC, USA). MCF10CA1a cells with luciferase label were generated in our previous study. 293T-eGFP cells, generated from ATCC HEK-293T cells, were a gift from Dr. Albert Cheng (Jackson Lab, USA). MOLM13 and NOMO1 cells stably expressing eGFP were generated by an infection with pCAG-eGFP lentivirus generated in HEK-293T cells cotransfected with packaging plasmids using Lipofectamin™ 2000 (plasmids from Addgene, USA) and sorted using flow cytometry. Leukemia and breast cancer cell lines were maintained in RPM11640 or DMEM (ThermoFisher Scientific) respectively with 10% fetal bovine serum (Biosera, USA) and 1% penicillin/streptomycin (ThermoFisher Scientific, USA). All the cells used for the experiments were tested negative for mycoplasma contamination using PCR. Briefly, 100 μl supernatants were collected from 80-100% confluent cultures and heated at 95° C. for 5 min. Mycoplasma DNA was amplified using Taq polymerase (a gift from the Xia lab, Xiamen University) and a mycoplasma-specific primer mixture (sequences provided below) in 35 cycles: 94° C. 15 s, 56° C. 15 s, 72° C. 30 s; and visualized using 1% agarose gel. In parallel, mycoplasma was also tested using the Mycofluor mycoplasma detection kit (ThermoFisher Scientific) according to the manufacturer's protocol. MOLM13, NOMO1, CA1a, and 293T cells were authenticated by their original vendors. The inventors also confirmed the identity of these cell lines and their fluorescent/luciferase derivatives using a 16-loci multiplex short tandem repeat analysis according to ATCC standards, provided by an authentication service by Guangzhou IGE Biotechnology (China).

### EV Purification From RBCs

Group O blood samples were obtained by Red Cross from healthy donors in Hong Kong with informed consents. All experiments with human blood samples were performed according to the guidelines and the approval of the City University of Hong Kong Human Subjects Ethics committee. RBCs were separated from plasma and white blood cells by using centrifugation and leukodepletion filters (Terumo Japan). Isolated RBCs were diluted in PBS and treated with 10 mM calcium ionophore (Sigma Aldrich) overnight. To purify EVs, RBCs and cell debris were removed by centrifugation at 600×g for 20 min, 1600×g for 15 min, 3260×g for 15 min, and 10,000×g for 30 min at 4° C. The supernatants were passed through 0.45 μm-syringe filters. EVs were concentrated by using ultracentrifugation with a TY70Ti rotor (Beckman Coulter, USA) at 100,000×g for 70 min at 4° C. EVs were resuspended in cold PBS. For labeling, half of the EVs were mixed with 20 pM PKH26 (Sigma Aldrich, USA) or 1 pM DiR (ThermoFisher Scientific) or 42.62 μM Vivo-Track-680 (Perkin Elmer). Labeled or unlabeled EVs were layered above 2 ml frozen 60% sucrose cushion and centrifuged at 100,000×g for 16 h at 4° C. using a SW41Ti rotor (Beckman Coulter) with reduced braking speed. The red layer of EVs (above the sucrose) was collected and washed once (unlabeled EVs) or twice (labeled EVs) with cold PBS using ultracentrifugation in a SW41Ti rotor (Beckman Coulter) at 100,000×g for 70 min at 4° C. All ultracentrifugation experiments were performed with a Beckman XE-90 ultracentrifuge (Beckman Coulter). Purified RBCEVs were stored at −80° C.

### EV Characterization

The concentration and size distribution of EVs were quantified using a NanoSight Tracking Analysis NS300 system (Malvern, UK). Zeta potential and polydispersity index were determined using a Zetasizer Nano(Malvern). For transmission electron microscopy analysis of EVs, EVs were fixed on copper grids (200 mesh, coated with formvar carbon film) by adding equal amount of 4% paraformaldehyde. After washing with PBS, 4% uranyl acetate was added for chemical staining of EVs and images were captured using a Tecnai 12 BioTWIN transmission electron microscope (FEI/Philips, USA).

### Oligonucleotide Sequences and Modifications

Anti-miR-125b ASOs (5′-UCACAAGUUAGGGUCUCAGGGA-3) and negative control ASOs (5′-CAGUACUUUUGUGUAGUACAA-3) were synthesized with 2′ Omethyl modification at every ribonucleotide by Shanghai Genepharma (Shanghai, China) or by Integrated DNA Technology (Singapore). Anti-miR 125b gRNA (5′-CCUCACAAGUUAGGGUCUCA-Synthego Scaffold-3′) and anti-GFP gRNA: (5′-GGGCACGGGCAGCUUGCCGG-Synthego Scaffold-3) were synthesized with 2′-O-methyl analogs and 3′ phosphorothioate internucleotide linkages at the first three 5′ and 3′ terminal RNA residues by Synthego (USA).

### EV Electroporation

Electroporation of RBCEVs were performed using a Gene Pulser Xcell electroporator (BioRad), exponential program at a fixed capacitance of 100 μF with 0.4 cm cuvettes. For optimization, 8.25 to 16.5×1011 RBCEVs were diluted in OptiMEM (ThermoFisher Scientific) and mixed with 4 μg Dextran conjugated with Alexa Fluor® 647 (AF647, ThermoFisher Scientific) to a total volume of 200 μl. An aliquot of 100 μl EV mixture was added to each cuvette and incubate on ice for 10 min. Electroporation was tested at different voltages: 50-250V. For ASOs delivery, ˜4-16×1011 RBCEVs were electroporated with 400 pmol scrambled negative control (NC) or anti-miR 125b ASOs at 250 V. For genome editing, 12.4×1011 RBCEVs (or MOLM13 cells) were electroporated with 6 pmol CleanCap™ Cas9 mRNA (Trilink) and 50 pmol anti-GFP gRNA or 80 pmol anti-mir-125b gRNA at 400 V. Aggregates of RBCEVs formed during electroporation were dissolved by vigorous pipetting. To quantify the electroporation efficiency, 8.25×1011 Dextran-AF647 electroporated EVs were incubated overnight with 5 μg latex beads (ThermoFisher Scientific) and analyzed for AF647 using flow cytometry.

### RNA Loading Efficiency and Stability in Electroporated RBCEVs

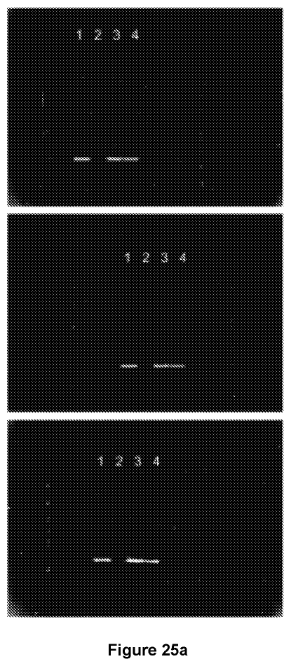

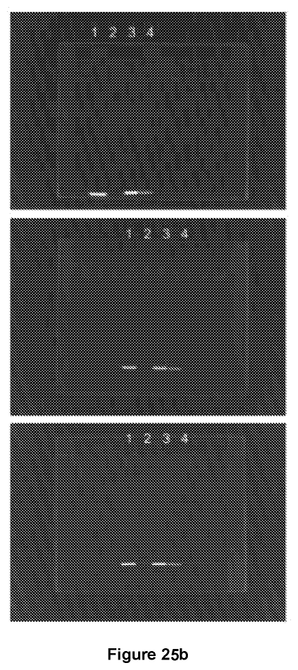

To quantify the amount of unbound ASOs, 200 pmol unlabeled or FAM labeled NCASOs were loaded with or without 8.25×1011 RBCEVs, with or without electroporation, into 10% Tris-acetate-EDTA (TBE) native gel, separated at 150 V for 30 min and visualized using SYBR-Gold staining for 30 min at room temperature (Thermo-Fisher Scientific) or using FAM fluorescence, respectively with the Gel Doc™ EZ Documentation system (Bio Rad, USA). The experiment was repeated three times independently. The SYBR Gold bands of the ASOs were quantified using imageJ (NIH, USA) and normalized to the background. Full images of the gels are provided in FIG. 25. To determine the stability of ASOs in electroporated EVs, 6.2×1011 of RBCEVs and 200 pmol FAM ASOs unelectroporated or electroporated mixtures were incubated with 50% FBS in OptiMEM at 37° C. for 1-72 h. The mixtures were added into a 96-well black plate with clear bottom (Perkin Elmer, USA) and FAM fluorescence was analyzed using a Synergy™ H1 microplate reader (BioTek, USA). To quantify the efficiency of ASOs electroporation, 6.2×1011 of RBCEVs electroporated with 200 pmol of 125b-ASOs or the same amount of unelectroporated EVs and 125b-ASOs were incubated with 100 units of RNase If (New England Biolabs, USA) at 37° C. for 30 min. The RNase was heat inactivated by incubating at 70° C. for 10 min. Trizol-LS (ThermoFisher Scientific) was added into each sample and the extracted RNA was reverse transcribed as described below. Similarly, 6.2×1011 RBCEVs electroporated with 1 μg of Cas9 mRNA or the same amount of unelectroporated Cas9 mRNA were incubated with 25 units of RNaself at 37° C. for 5 min and subjected to RNA extraction and qRT-PCR of Cas9 mRNA.

### EV Separation Using Top-Down Sucrose Density Gradients

A total of 3.7×1012 FAM ASOs-loaded EVs were mixed with 1 ml of 10% HEPES/sucrose solution. An 11 ml of linear sucrose gradient (60-10%) was loaded into a 12.5 ml-open top SW41 ultracentrifuge tube (Beckman Coulter). The EV suspension was layered on top of the sucrose layer and ultracentrifuged at 150,000×g for 16 h at 4° C. A total of 12 fractions were collected from the sucrose gradient into a black well plate and analyzed using the Synergy™ H1 Microplate Reader. The concentration of EVs in each fraction was determined using the NanoSight as described above. The density of sucrose in each fraction was measured using a refractometer (VastOcean, China).

Treatment of Cancer Cells With RBCEVs in vitro

We performed quality control of RBCEVs for every batch of purification using a Nanosight particle analyzer. Samples with unusually low concentration or strange aggregates were discarded. Cells in culture were routinely examined for signs of contamination or changes in morphology and growth. To test the EV uptake, 50,000 MOLM13, CA1a or NOMO1 cells were incubated with 200 μl of ˜4-16×1011 unelectroporated or electroporated EVs and 300 μl growth medium per well in 24-well plates for 24 h. Untreated controls were kept in the same medium with 200 μl untreated Opti-MEM. For assays that required longer incubation time, the medium was replaced with fresh growth medium after 24 h. For comparison, MOLM13 cells were transfected with 4 μg Dextran-AF647 or 800 nM FAM-labeled NC-ASOs in Lipofectamin™ 3000 (ThermoFisher Scientific) or INTERFERin® (PolyPlus Transfection, France) according to the manufacturers' protocols. To test the effect of heparin on the uptake, MOLM13 cells were pretreated with 20 μg/ml of heparin sodium salt (Aladdin, China) for 10 min then incubated overnight with 12.4×1011 of unlabeled or PKH26-labeled RBCEVs in the presence or absence of 20 μg/ml of heparin sodium salt. The uptake of RBCEVs and Dextran or FAM ASOs were analyzed using flow cytometry or immunostaining.

### Flow Cytometry Analysis

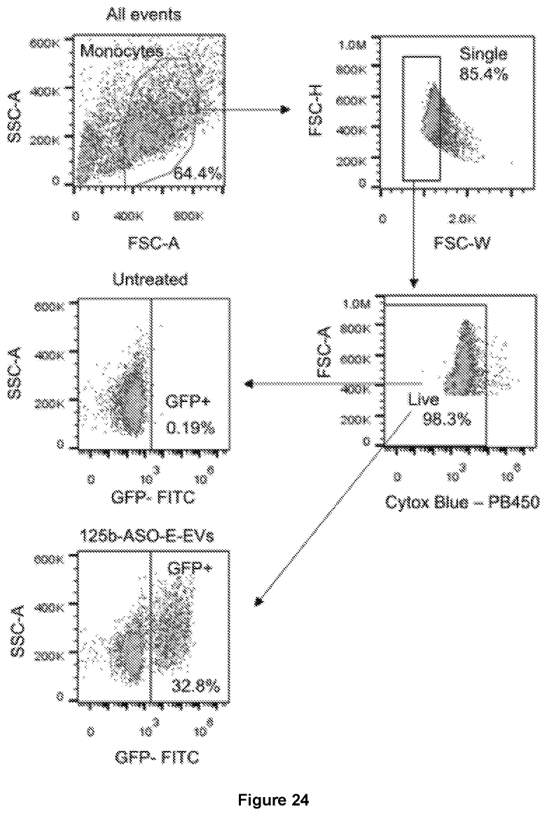

Flow cytometry of latex beads or cells in FACS buffer (PBS containing 0.5% fetal bovine serum) was performed using a CytoFLEX-S cytometer (Beckman Coulter) or SH800Z cytometer (Sony Biotechnology, USA) and analyzed using Flowjo V7 or V10 (Flowjo, USA). GFP-positive MOLM13 or NOMO1 cells were selected using a Sony SH800Z cell sorter in sterile condition. The beads or cells were initially gated based on FSC-A and SSC-A to exclude the debris and dead cells (low FSC-A) as shown in FIGS. 11 & 24. The cells were further gated based on FSC-width vs. FSC-height, to exclude doublets and aggregates. In the analysis of GFP+cells from the bone marrow of leukemic NSG mice, the live cells were also gated from the single cells population based on Cytox blue negative (PB450 channel). Subsequently, the fluorescent-positive beads or cells were gated in the appropriate fluorescent channels: FITC for FAM and GFP, PE for PKH26 and PI, APC for AF647, and APCCy5.5 for VVT, as the populations that exhibited negligible signals in the unstained/untreated negative controls as shown by the example in FIGS. 11, 24.

### Western Blot Analysis

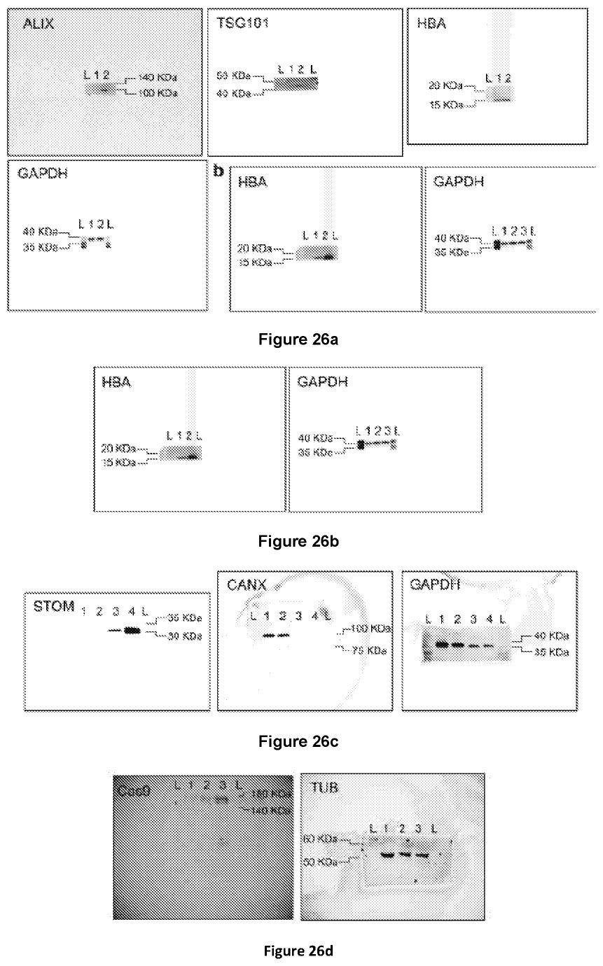

Total cell lysates were extracted from EVs or cells (cells from 3 wells of 24-well plates were combined for each condition) by incubating with RIPA buffer supplemented with protease inhibitors (Biotool). A total of 30 or 35 μg of protein lysates were separated on 10% polyacrylamide gels and transferred to a Nitrocellulose membrane (GE Healthcare). PM5100 ExcelBand™ 3-color high range protein ladder (SmoBio, Taiwan) was loaded at two sides of the samples. Membranes were cut horizontally into two to four pieces based on the ladder, blocked with 5% non-fat milk in Tris buffered saline containing 0.1% Tween-20 (TBST) for 1 h at room temperature and incubated with primary antibodies overnight at 4° C.: mouse anti-Alix (clone 3A9, Cat # SC-53538, Santa Cruz, dilution 1:500), mouse anti-Tsg101 (clone C-2, Cat # SC-7964, Santa Cruz, dilution 1:500), rabbit anti-Hemoglobin α (clone H-80, SC-21005, Santa Cruz, dilution 1:1000), mouse anti-Calnexin (clone AF18, SC-23954, Santa Cruz, dilution 1:500), mouse anti-Stomatin (clone E-5, SC-376869, Santa Cruz, dilution 1:1000), rabbit anti-GAPDH (clone FL-335, Cat # SC-25778, Santa Cruz, dilution 1:1000), mouse anti-Cas9 antibody (clone 7A9, Cat # 844301, BioLegend, dilution 1:250), and mouse anti-tubulin (clone DM1A, Cat # Ab7291, Abcam, dilution 1:1000). The blot was washed three times with TBST then incubated then with HRP-conjugated anti-mouse and anti-rabbit secondary antibodies (Cat # 715-035-150 and 711-035-152, Jackson ImmunoResearch, dilution 1:10,000,) for 1 h at room temperature. The blot was imaged using an Azure Biosystems gel documentation system. Full images of the blots are provided in FIG. 26. The intensity of the bands were quantified and subtracted by the background using ImageJ.

RNA Extraction and qRT-PCR

Total RNA was extracted from cells or tissues using Trizol (ThermoFisher) according to the manufacturer's manuals. RNA samples were quantified and qualified using NanoDrop analysis (ThermoFisher) and 1% agorose gels. Those with sufficient concentration (>30 ng/μl), purity (A260/280>1.7) and integrity (clear 28 and 18s rRNA bands) were converted to cDNA using a high capacity cDNA reverse transcription kit (ThermoFisher) following the manufacturer's protocol. The levels of miR-125a and miR-125b were quantified using Taqman® miRNA assays (ThermoFisher), normalized to the expression of U6b snRNA, or using the miRCURY™ LNA™ Universal RT microRNA PCR (Qiagen, Germany), normalized to the expression of miR-103a. Anti-miR-125b ASOs was quantified using a Taqman® miRNA assay (ID #007655_mat), normalized to the expression of U6b snRNA or its copy number was calculated based on a standard curve of ASOs threshold cycle (Ct) values vs. the concentration of the ASOs in a serial dilution from 0.001 to 1000 pM. qRT-PCR analysis of mRNAs was performed using Ssofast™ Evagreen (SYBR Green) qPCR master mix (Bio-Rad), normalized to the expression of GAPDH, or 18s rRNA, or ACTB. All qPCR reactions were performed by using a CFX96 Touch™ Real-Time PCR Detection System (Bio-Rad).

Sequencing the mir-125b Gene

To identify the genome editing events generated by mir-125b targeting gRNA and Cas9 mRNA-loaded RBCEVs, DNA were extracted from the EV treated MOLM13 cells using Trizol followed by phenol-chloroform and ethanol precipitation. The primary hsa-mir-125b-2 sequence (383 nucleotides) was amplified using a pair of gene-specific primers (sequences provided below) and Q5® Hot Start high-fidelity master mix (New England Biolabs) in 30 cycles: 98° C. 10 s, 64° C. 30 s, 72° C. 45 s, and a final extension of 72° C. for 2 min. The PCR product was reamplified using the same primers with sequencing tags. High-through sequencing was performed by NovoGene Sequencing company (Hong Kong) using the HiSeq paired-end platform (Illumina, USA).

Primer sequences
GAPDH Forward:
GGAGCGAGATCCCTCCAAAAT
GAPDH Reverse:
GGCTGTTGTCATACTTCTCATGG
18s RNA Forward:
GTAACCCGTTGAACCCCATT
18s RNA Reverse:
CCATCCAATCGGTAGTAGCG
BAK1 Forward:
TGGTCACCTTACCTCTGCAA
BAK1 Reverse:
TCATAGCGTCGGTTGATGTC
SP-Cas9 Forward:
AAGGGACAGAAGAACAGCCG
SP-Cas9 Reverse:
ATATCCCGCCCATTCTGCAG
mir-125b Forward:
AATGGTCGTCGTGATTACTCA
mir-125b Reverse:
TTTTGGGGATGGGTCATGGT
ACTB Forward:
TCCCTGGAGAAGAGCTACGA
ACTB Reverse:
AGGAAGGAAGGGTGGAAGAG
Mycoplasma-1 Forward:
CGCCTGAGTAGTACGTTCGC
Mycoplasma-2 Forward:
CGCCTGAGTAGTACGTACGC
Mycoplasma-3 Forward:
TGCCTGAGTAGTACATTCGC
Mycoplasma-4 Forward:
TGCCTGGGTAGTACATTCGC
Mycoplasma-5 Forward:
CGCCTGGGTAGTACATTCGC
Mycoplasma-6 Forward:
CGCCTGAGTAGTATGCTCGC
Mycoplasma-1 Reverse:
GCGGTGTGTACAAGACCCGA
Mycoplasma-2 Reverse:
GCGGTGTGTACAAAACCCGA
Mycoplasma-3 Reverse:
GCGGTGTGTACAAACCCCGA

All primers were synthesized by Beijing Genomics Institute (China).

### Quantification of Cell Viability

For flow cytometry analysis, leukemia cells were stained with Propidium iodide (PI) for 15 min at room temperature, washed with FACS buffer and analyzed using a CytoFLEX-S cytometer as described above. For a plate assay, 50,000 CA1a cells were seeded per well in 24-well plates and treated with ASO-electroporated RBCEVs. After 3 days, the cells were washed once with PBS, and incubated with 0.5% crystal violet staining solution (Sigma Aldrich). Plates were then washed three times in a stream of tap water and air dried. Afterwards, 500 μl of 50% acetic acid was added into each well and the optical density was measured at 570 nm using the Biotek micro-plate reader.

### Animal Experiments

All mouse experiments were performed according to experimental protocols approved by the Animal Ethics Committees at City University of Hong Kong and the Department of Health, Hong Kong SAR, complied with the government legislations including the Animals (Control of Experiments) Ordinance (Cap. 340) and the Prevention of Cruelty to Animals Ordinance (Cap.169). Nude mice (strain NU/J 002019) and NSG mice (NOD.CgPrkdc<scid>ll2rg <tm1Wjl>/SzJ 005557) were purchased from the Jackson Laboratory (USA) and bred in our facilities. Mice of similar ages were labeled with numbers on their ear tags or tails using permanent markers and randomized into control and treatment groups without any bias on parents, weight, size, or gender (except for the breast cancer experiments that were performed in female mice only). Most of the imaging and histopathology experiments were done in a blinded fashion as the researchers who performed the data collection were different from those who performed the treatments. The data was recorded based on the mouse numbers, not by their treatment groups. Mice that became pregnant or died accidentally due to anesthesia during the experiments were excluded. False positives or negatives due to other technical issues such as unsuccessful injections were also excluded.

Determination of EV Uptake and Biodistribution in vivo

A total of 5×106 CA1a cells in 50 pl PBS were mixed with an equal volume of cold Matrigel (Corning) and injected subcutaneously in the left and right flanks of 6-weekold female nude mice. After 1 week, when the tumors approach 7 mm in diameter, each mouse was injected with 16.5×1011 PKH26-labeled RBCEVs intratumorally in the left flank. The mice were subsequently fed with alfalfa-free diet (LabDiet, USA) to reduce autofluorescence in the digestive system. PKH26 fluorescence was measured every day using the IVIS Lumina III system (Caliper Life Sciences, USA). On day 3, mice were killed and the tumors were excised and imaged for PKH26. Frozen sections of the tumors were stained with DAPI and observed under a LSM-880 NLO confocal laser scanning microscope (Zeiss, Germany). Similarly, the CA1a tumor (7 mm) bearing mice were injected i.p. with 16.5×1011 PKH26 or DiR-labeled RBCEVs or the supernatant of the last wash after EV labeling. Images of DiR EV injected mice were captured at 24 h post-treatment using the IVIS Lumina III. Tumors, livers, hearts, lungs, spleens, kidneys, stomach, and intestines were also obtained from i.p. DiR/PKH26-EV-injected mice for IVIS imaging and histopathology analysis.

To determine the biodistribution of RBCEVs in NSG mice, 3.3×1012 DiR-labeled RBCEVs or the supernatant of the last EV wash were injected twice with 24h interval in 6-8-week-old NSG mice (Jackson Lab). Thereafter, the mice were killed and the organs were collected for DiR fluorescence imaging using the IVIS Lumina III system. Whole body and tissue fluorescence images were acquired and analyzed using Living Image® software (Caliper Life Sciences). The background and autofluorescence were defined using the untreated or supernatant negative controls and subtracted from the images using Image-Math function.

To determine the uptake of RBCEVs by cells in the bone marrow, 3.3×1012 VVTlabeled RBCEVs or the supernatant of the last EV wash were injected twice with 24 h intervals in 6-8-week-old NSG mice (4 mice/group). 24 h after the second injection, the mice were killed and their bone marrow was collected by flushing FACS buffer through the crushed bones with a syringe and G25 needle. The cells were filtered through a 70 μm strainer, centrifuged at 1500 rpm for 5 min, resuspended in FACS buffer and analyzed for VVT fluorescence (APC-Cy5.5 channel) using the Sony SH800Z cytometer as described above.

### Stability of RBCEVs in the Circulation

A total of 3.3×1012 PKH26-labeled EVs were injected i.v. into the tail veins of 6-week NSG mice (Jackson Lab). The mice were killed immediately or 3, 6, and 12 h after the injection. The blood was collected from the heart into the EDTA tubes and centrifuged at 800×g for 5 min. The supernatant was diluted with 10 ml cold PBS and centrifuged at 10,000×g for 15 min and passed through a 0.45 μm filter to remove the debris. Afterwards, the EVs were ultracentrifuged at 100,000×g for 90 min at 4° C. Purified EVs were resuspended in 175 μl PBS and incubated with 2.5 μg of latex beads at 37° C. for 30 min, followed by an overnight incubation at 4° C. EVbound latex beads were washed with 1 ml FACS buffer at 4000×g for 10 min and PKH26 fluorescence was analyzed using the CytoFLEX-S cytometer (Beckman).

In vivo Delivery of ASOs to Breast Tumors

A total of 5×105 luciferase-labeled CA1a cells were mixed with equal volume of matrigel and injected into the flanks of 6-week-old female nude mice. After 24 h, each mouse was injected with 8.25×1012 NC/125b-ASO-loaded (n=8 mice) or 400 pmol unbound NC/125b-ASOs (n=6 mice) subcutaneously in the flank at the same site where the tumor cells were injected. Intratumoral injections of the ASOelectroporated EVs were repeated every 3 days until day 42. Bioluminescent images of the whole body were taken every 6 days using the IVIS Lumina III system following i.p. injection of 150 mg/kg D-luciferin (Caliper Life Sciences). All bioluminescent images were acquired and analyzed using Living Image® 4.5. software in a blinded manner (Caliper Life Sciences). On day 44, the mice were killed and the tumors were excised. Half of each tumor was homogenized in Trizol for RNA extraction and qRT-PCR of miR-125b, except the RNA samples from a few tumors that did not meet the quality controls (as described above). The other half of the tumor and the lung was fixed for histopathology analysis as described below.

In vivo Delivery of ASOs to Leukemia Engrafted Mice

NSG mice of 7-8-week-old were injected i.p. with 20 mg/kg Busulfan (Santa Cruz). After 24 h, 5×105 luciferase and GFP labeled MOLM13 cells were injected into the tail vein of the busulfan-conditioned mice. A week later, bioluminescence was measured using the IVIS Lumina III. Mice with successful engraftment of leukemia cells (shown by bioluminescent signals in the bone marrow) were treated with 3.3×1012 ASO-loaded EVs i.p. every other day for 9 days (5 doses in total). Luminescence images of whole body were taken every 3 days using the IVIS Lumina III system following i.p. injection of 150 mg/kg D-luciferin in a blinded manner (Caliper Life Sciences). After 9 days of treatment, the mice were killed. Bone marrow were collected and analyzed for GFP-positive cells using FACS. Briefly the cells were collected from the femur or tibiae of killed mice by flushing FACS buffer through the crushed bones with a syringe and G25 needle. The cells were filtered through a 70 μm strainer, centrifuged at 1500 rpm for 5 min, resuspended in 0.5 ml RBC lysate buffer (0.8% NH4Cl and 0.1 mM EDTA in water buffered with KHCO3 to achieve a final pH of 7.2-7.6) and incubated on ice for 5 min. The buffer was neutralized with 5 ml DMEM containing 10% FBS. The cells were centrifuged again, washed once with PBS and resuspended in 200 pl FACS buffer. 0.5 pl Cytox blue (ThermoFisher Scientific) was added for identification of dead cells. The spleen and liver were collected in Trizol or formalin for RNA extraction and histopathology analysis, respectively. The RNA samples were used for qRT-PCR of miR-125b, except those that did not meet the quality controls (as described above).

### Histopathology

Tumors and other tissues from the mice were fixed in 10% buffered formalin (ThermoFisher Scientific) overnight at room temperature, dehydrated sequentially in 70, 95, and 100% alcohol at 37° C., cleared in three baths of xylene (ThermoFisher Scientific) and impregnated in three baths of paraffin wax (ThermoFisher Scientific) each for 1 h and 30 min at 37 and 62° C., respectively. Paraffin blocks were cut at 5 μm using a microtome (MICROM model: HM330). Sections were dried in a 37° C. incubator before staining. Sections were dewaxed in two baths of xylene, then immersed in two baths of absolute alcohol and one bath of 70% alcohol, each for 10 min. Sections were rehydrated in water and stained with Gill's haematoxylin (Surgipath) for 15 min. After washing with water, stained sections were differentiated in 0.3% acid alcohol, washed in water again and blued in 2% sodium bicarbonate solution. Microscopic examination is essential to check distinct nuclei staining with clean cytoplasm and background. Sections were then stained with 0.5% Eosin for 2 min. After a quick wash in water to remove excess Eosin (Merck), sections were dehydrated in 95% and absolute alcohol. Sections were then cleared in xylene and mounted with a synthetic mountant (Shandon).

### Immunostaining

MOLM13 cells were fixed with 4% paraformaldehyde (Sigma Aldrich) and adhered to glass slides using cytospin at 400 rpm for 3 min. The cells were blocked with 5% normal donkey serum (Jackson Immuno Research), permeabilized with 0.2% Triton X-100, and incubated overnight with a mouse anti-HA antibody (Clone F-7, Cat #7392, Cell Signaling Technology, 1:250 dilution) at 4° C., washed three times with PBS and incubated with Alexa Fluor® 488 donkey anti-mouse secondary antibody (Jackson Immuno Research) for 1 h at room temperature. The cells were finally stained with Hoechst (Sigma Aldrich) for 5 min at room temperature and washed three times with PBS. In the EV uptake experiment (FIG. 10, the cells were stained with Hoechst only but not with any antibody. Images were captured using Nikon Eclipse Ni-E upright fluorescence microscope. Images were analyzed using ImageJ. The number of Cas9-HA positive nuclei were counted and normalized by the number of Hoechst positive nuclei in the same image. The average percentage of Cas9-HA positive cells was calculated from three samples in each treatment.

### Statistical Analysis

Student's t-tests, calculated using Microsoft Excel, were used to compute the significance between the treated samples and the controls; the test type was set to one-tail distribution and two-sample equal variance. The Mann-Whitney test (onetail), computed using GraphPad Prism 6, was employed to calculate the significance where the data did not follow a normal distribution. One-way ANOVA, calculated using GraphPad Prism 6, was used for analysis of data involving multiple groups of treatments. All P-value<0.05 were considered significant. In all graphs, data are presented as mean±standard error of the mean (SEM). For quantification, each experiment was usually repeated three times with RBCEVs from three donors or with cells from three cell passages. Mouse experiments were performed with groups of 3 to 8 mice. The minimum sample size of 3 was determined using G*Power analysis for one-tail t-test comparing the mean difference of two independent groups with effect size d=5; α err prob=0.05 and power=0.95.

### REFERENCES

A number of publications are cited above in order to more fully describe and disclose the invention and the state of the art to which the invention pertains. Full citations for these references are provided below. The entirety of each of these references is incorporated herein.
1. Pitt, J. M., Kroemer, G. & Zitvogel, L. Extracellular vesicles: masters of intercellular communication and potential clinical interventions. J. Clin. Invest. 126, 1139-1143 (2016).
2. Kamerkar, S. et al. Exosomes facilitate therapeutic targeting of oncogenic KRAS in pancreatic cancer. Nature 546, 498-503 (2017).
3. Syn, N. L., Wang, L., Chow, E. K.-H., Lim, C. T. & Goh, B.-C. Exosomes in Cancer Nanomedicine and Immunotherapy: Prospects and Challenges. Trends Biotechnol. (2017). doi:10.1016/j.tibtech×.2017.03.004
4. Usman, W. et al. Efficient RNA drug delivery using red blood cell extracellular vesicles. Nature Communications In press, (2018).
5. Vader, P., Breakefield, X. O. & Wood, M. J. A. Extracellular vesicles: emerging targets for cancer therapy. Trends in Molecular Medicine 20, 385-393 (2014).
6. Kooijmans, S. A. A., Gitz-Francois, J. J. J. M., Schiffelers, R. M. & Vader, P. Recombinant phosphatidylserine-binding nanobodies for targeting of extracellular vesicles to tumor cells: a plug-and-play approach. Nanoscale 10, 2413-2426 (2018).
7. Shi, J. et al. Engineered red blood cells as carriers for systemic delivery of a wide array of functional probes. Proc. Natl. Acad. Sci. U.S.A. 111, 10131-10136 (2014).
8. Pishesha, N. et al. Engineered erythrocytes covalently linked to antigenic peptides can protect against autoimmune disease. Proc. Natl. Acad. Sci. U.S.A. 114, 3157-3162 (2017).
9. Fridy, P. C. et al. A robust pipeline for rapid production of versatile nanobody repertoires. Nat Methods 11, 1253-1260 (2014).
10. Rodriguez, P. L. et al. Minimal ‘Self’ Peptides That Inhibit Phagocytic Clearance and Enhance Delivery of Nanoparticles. Science 339, 971-975 (2013).
11. Li, Z. et al. Identification and characterization of a novel peptide ligand of epidermal growth factor receptor for targeted delivery of therapeutics. FASEB J. 19, 1978-1985 (2005).
12. Yang, R. et al. Engineering a Catalytically Efficient Recombinant Protein Ligase. J. Am. Chem. Soc. 139, 5351-5358 (2017).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An extracellular vesicle derived from a red blood cell and containing an exogenous cargo.

2. The extracellular vesicle according to claim 1 wherein the cargo is a nucleic acid, peptide, protein or small molecule.

3. The extracellular vesicle according to claim 1 or claim 2 wherein the cargo is a nucleic acid selected from the group consisting of an antisense oligonucleotide, a messenger RNA, a long RNA, a siRNA, a miRNA, a gRNA or a plasmid.

4. The extracellular vesicle according to claim 3, wherein the nucleic acid comprises one or more modifications, non-naturally occurring elements or non-naturally occurring nucleic acids.

5. The extracellular vesicle according to claim 4, wherein the one or more modifications, non-naturally occurring elements or non-naturally occurring nucleic acids is selected from a 2′-O-methyl analog, a 3′ phosphorothioate internucleotide linkage or other locked nucleic acid (LNA), an ARCA cap, a chemically modified nucleic acids or nucleotides or a 3′ or 5′ modification such as capping.

6. The extracellular vesicle according to claim 5 wherein the non-naturally occurring nucleotide is selected from a 2′-position sugar modification, 2′-O-methylation, 2′-Fluoro modification, 2′NH2 modification, 5-position pyrimidine modification, 8-position purine modification, a modification at an exocyclic amine, substitution of 4-thiouridine, substitution of 5-bromo, or 5-iodo-uracil, or a backbone modification.

7. An extracellular vesicle according to claim 1 wherein the cargo comprises one or more components of a CRISPR×/Cas9 gene editing system.

8. An extracellular vesicle according to claim 7 wherein the cargo is a nuclease, or an mRNA or plasmid encoding a nuclease.

9. The extracellular vesicle according to claim 7 wherein the cargo comprises a gRNA.

10. An extracellular vesicle according to claim 1 comprising an antisense nucleic acid.

11. A composition comprising one or more extracellular vesicles according to any one of claims 1-10.

12. An extracellular vesicle or composition according to any one of the preceding claims, for use in a method of treatment.

13. A method of treatment, the method comprising administering an extracellular vesicle according to claim 1 to a patient in need of treatment.

14. Use of an extracellular vesicle or composition according to any one of claims 1-11 in the manufacture of a medicament for the treatment of a disease or disorder.

15. Use of an extracellular vesicle or composition according to any one of claims 1-11 in the manufacture of a medicament for the treatment of a disease or disorder.

16. The extracellular vesicle or composition for use, method of treatment or use according to any one of claims 10-12 wherein the method of treatment involves administration of an extracellular vesicle or composition according to any one of claims 1-9 to a subject with a genetic disorder, inflammatory disease, cancer, autoimmune disorder, cardiovascular disease or a gastrointestinal disease.

17. The extracellular vesicle or composition for use, method of treatment or use according to claim 13 wherein the subject has cancer, the cancer optionally selected from leukemia, lymphoma, myeloma, breast cancer, lung cancer, liver cancer, colorectal cancer, nasopharyngeal cancer, kidney cancer or glioma.

18. A method comprising:
a. providing a sample of red blood cells, wherein said sample is free from leukocytes;
b. contacting the sample of red blood cells with a vesicle inducing agent;
c. separating extracellular vesicles from red blood cells; and
d. collecting the extracellular vesicles.
e. optionally electroporating the extracellular vesicles in the presence of an exogenous cargo.

19. A method comprising electroporating an extracellular vesicle derived from a red blood cell in the presence of an exogenous cargo.

20. A composition comprising extracellular vesicles obtained by the method according to claim 18 or claim 10.
